(19) United States
(12) Patent Application Publication
(10) Pub. No.: US 2007/0066616 A1
(43) Pub. Date: Mar. 22, 2007

(54) Inhibitors for extracellular signal-regulated kinase docking domains and uses therefor

(76) Inventors: Paul Shapiro, Baltimore, MD (US); Alexander D. MacKerell JR., Baltimore, MD (US)

Correspondence Address: Benjamin Aaron Adler;ADLER & ASSOCIATES, 8011 Candle Lane, Houston, TX, 77071, US

(21) Appl. No.: 11/543,491

(22) Filed: Oct. 5, 2006

Related U.S. Application Data
(63) Continuation-in-part of application No. PCT/US06/11536, filed on Mar. 29, 2006.
(60) Provisional application No. 60/666,206, filed on Mar. 29, 2005.

(51) Int. Cl. A61K 31/5375; A61K 31/495; A61K 31/427; A61K 31/426; A61K 31/423; A61K 31/165

(52) U.S. Cl. 514/237.5; 514/255.03; 514/369; 514/370; 514/375; 514/617

(57) ABSTRACT

Provided herein are compounds and methods of using compounds that selectively inhibit binding to one or more docking domain regions of an extracellular signal-regulated kinase (ERK) to inhibit in a cell having an extracellular signal-regulated kinase activity. Such methods may be used to inhibit cell proliferation of a neoplastic cell, to treat a cancer and further may be used in conjunction with administration of an anticancer drug at a reduced dosage to treat a cancer with a concomitant reduction in toxicity to an individual receiving the treatment. Also provided is a method to design and screen for compounds to inhibit binding within the extracellular signal-regulated kinase docking domain region, using at least in part computer-aided drug design modeling.

### CROSS-REFERENCE TO RELATED APPLICATIONS

This is a continuation-in-part of International Application PCT/US2006/011536, with an international filing date of Mar. 29, 2006, which claims priority to provisional application U.S. Ser. No. 60/666,206, filed Mar. 29, 2005, now abandoned.

### FEDERAL FUNDING LEGEND

This invention was produced in part using funds obtained through grants CA105299-01, CA95200-01 and CA095200-03S1 from the National Institutes of Health. Consequently, the federal government has certain rights in this invention.

### BACKGROUND OF THE INVENTION

1. Field of the Invention

The present invention relates generally to the fields of enzymology, computer-aided drug design and screening and oncology. More specifically, the present invention relates to specific inhibitors of extracellular signal-regulated kinase (ERK) docking domains useful in the treatment of cancer.

2. Description of the Related Art

Mitogen activated protein (MAP) kinases consist primarily of the extracellular signal regulated kinases 1 and 2 (ERK1/2), c-Jun N-terminal kinases (JNK), and p38 MAP kinases (1). MAP kinases play a central role in the regulation of most biological processes including cell growth, proliferation, differentiation, inflammatory responses and programmed cell death. Unregulated activation of MAP kinases has been linked to cancer cell proliferation and tissue inflammation (2-5).

Activation of ERK proteins most often occurs through a process where a ligand-activated plasma membrane receptor facilitates the sequential activation of the Ras G-proteins, Raf kinases, and the MAP or ERK kinases-1 and 2 (MEK1/2), which are the only known activators of ERK1 and ERK2 (7). The activation of ERK proteins by MEK1/2 is regulated by direct phosphorylation of threonine (Thr) 183 and tyrosine (Tyr) 185, where the amino acid numbering is according to mouse sequence, accession #P63085, where phosphorylation of both sites is required for full activation. Once ERK is phosphorylated, it undergoes structural changes that are important for phosphoryl transfer onto substrate proteins (8).

In vitro studies suggest that active ERK proteins may phosphorylate more than 50 different substrates (1,7). However, it is not clear whether all of these substrates are physiological targets in vivo or whether activated ERK selectively phosphorylates specific substrates in response to a particular extracellular signal. Importantly, hyper-activation of the ERK MAP kinases has been linked to unregulated cell proliferation in cancer cells. For example, naturally occurring mutations in Ras and Raf proteins, which cause hyper-activation of the ERK pathway, are found in almost 30% of all human cancers (3,9- 10).

The mechanisms involved in determining the interactions between the ERK proteins and their cognate substrate proteins are still largely unknown. Similarly, it is not clear how ERK distinguishes between its own protein substrates and substrates that are phosphorylated by the JNK or p38 MAP kinases. Studies in recent years have revealed at least three protein motifs that provide clues as to how ERK proteins interact with and phosphorylate specific substrate proteins.

First, ERK proteins are proline directed serine or threonine kinases that prefer the consensus PXS/TP (X is any amino acid, P is proline, S is serine, and T is threonine) motif on the substrate protein (11). At a minimum, ERK proteins require a proline that is immediately C-terminal to the phosphorylated S or T residue. Second, ERK substrates may contain an FXFP (F is phenylalanine) motif, a D-domain containing basic residues followed by an LXL motif, or a kinase interaction motif (KIM), which are important for substrate interactions with ERK (12-13). Third, ERK proteins contain recently identified docking domains that have been shown to facilitate interactions with substrate proteins (14-16). The first identified ERK2 docking domains, referred to as common docking (CD) and ED domains, are positioned opposite the activation loop in the 3D crystallographic structure and appear to regulate the efficiency of substrate phosphorylation and interaction with the upstream MEK proteins (16). More recent data suggest that additional amino acid residues in the C-terminal domain of ERK2 may also form additional docking domains that regulate specific substrate interactions (14).

No specific inhibitors of the ERK proteins are currently available. Pharmacological inhibitors of Ras G-proteins, Raf kinases, and MEK1/2 have been used successfully to block the ERK pathway and are being tested in cancer clinical trials (17-20). Since ERK proteins are involved in many cellular functions, it may be more beneficial to selectively block ERK involvement in abnormal cell functions, such as cancer cell proliferation, while preserving ERK functions in regulating normal metabolic processes. Given that most kinase inhibitors lack specificity because they compete with ATP binding domains that are conserved among protein kinases (6,21), it is contemplated that small molecular weight compounds that interact with specific ERK docking domains can be used to specifically disrupt ERK2 interactions with protein substrates. Recent successes in CADD approaches in the identification of inhibitors of protein-protein interactions (23-26), indicated that such an approach was feasible for identification of inhibitors specific to ERK.

There is a need in the art for improvements in the development of specific small molecular weight MAP kinase inhibitors as an effective approach towards the identification of chemotherapeutic and anti-inflammatory agents. Specifically, the prior art is deficient in inhibitors that block extracellular signal-regulated kinase docking domains. The present invention fulfills this long-standing need and desire in the art.

### SUMMARY OF THE INVENTION

The present invention is directed to a method of inhibiting an activity of an extracellular signal-regulated kinase in a cell. The method comprises contacting the cell with an inhibitory compound that selectively binds to one or more docking domain regions of the ERK thereby inhibiting an ERK activity associated with an ERK substrate binding thereto.

The present invention also is directed to a method of inhibiting proliferation of a neoplastic cell. The neoplastic cell is contacted with an inhibitory compound that selectively inhibits binding of a substrate of an extracellular signal-regulated kinase to one or more docking domain regions thereof whereby proliferation of the neoplastic cell is inhibited. The inhibitory compound may be compound 17, compound 76, compound 86, compound 89, compound 92, compound 93, compound 94, or compound 95.

The present invention is directed further to a method of treating a cancer in a subject. The method comprises administering an inhibitory compound that selectively binds one or more docking domain regions of an extracellular signal-recognition kinase. Reducing proliferation of the cancer cells treats the cancer. The method may comprise a further step of administering an anticancer drug to the individual.

The present invention is directed to a related method of reducing toxicity of a cancer therapy in an individual in need thereof. The method comprises co-administering to the individual an inhibitory compound that selectively binds one or more docking domain regions of an extracellular signal-recognition kinase and an anticancer drug. The dosage of the anticancer drug administered with the inhibitor is lower than a dosage required when the anticancer drug is administered singly. Toxicity of the cancer therapy to the individual is thereby reduced.

The present invention is directed further still to a method of identifying an inhibitor of substrate binding to a docking domain region of an extracellular signal-reduction kinase. A test compound that binds one or more docking domain regions in the extracellular signal-regulated kinase, but does not interfere with the ATP binding domain, is designed based at least in part, computer-aided drug design (CADD) modeling. Inhibitory efficacy is determined by measuring the level of phosphorylation of a ERK substrate protein in the presence or absence of the test compound and comparing the level of protein phosphorylation in the presence of the test compound with the level of protein phosphorylation in the absence of the test compound. A decrease in protein phosphorylation in the presence of the test compound is indicative that the test compound is an inhibitor of binding to one or more docking domain regions in ERK.

The present invention is directed to a further method of screening the inhibitor for anti-cell proliferative activity directed against neoplastic cells. A culture of the neoplastic cells having an activated ERK activity is contacted with the inhibitor and the amount of cell proliferation of the neoplastic cells in the presence of the inhibitor is compared with the amount of cell proliferation of the neoplastic cells in the absence of the inhibitor. A decrease in cell proliferation in the presence of the inhibitor compared to cell proliferation in the absence of the inhibitor is indicative that the inhibitor has the ability to prevent cell proliferation in neoplastic cells.

The present invention is directed further still to inhibitory compounds identified by the screening methods described herein. These compounds inhibit binding one or more docking domain regions in ERK and thereby arrest proliferation of neoplastic cells. These compounds may be used in any of the methods of inhibiting cell proliferation of a neoplastic cell, of treating a cancer or of reducing toxicity of an anticancer drug described in the present invention.

The present invention is directed further to a related ERK inhibitory compound. The ERK inhibitory compound has a chemical structure comprising one or more substituted or unsubstituted heterocyclic aromatic ring moieties that are covalently coupled in a size and shape designed to bind one or more docking domain regions of an extracellular signal-reduction kinase without interfering with an ATP binding domain therein. The design of the synthetic compound is based at least in part on computer-aided drug design models. The present invention also is directed to a related ERK inhibitory compound. The substituted or unsubstituted heterocyclic aromatic ring moieties may be nitrogen, sulfur, or oxygen heteroatoms or a combination thereof and further have at least one of a pendant heteroatom, a pendant moiety having one or more heteroatoms, a side-chain having one or more heteroatoms or a combination thereof. The present invention also is directed further to the related ERK inhibitory compound that forms a bond with residues Asp316, Asp319 or a combination thereof comprising the CD domain and with at least one of residues Glu79, Asn80, Gln130, Arg133, Tyr314, Gln313 comprising the ED domain.

Other and further aspects, features, and advantages of the present invention will be apparent from the following description of the presently preferred embodiments of the invention. These embodiments are given for the purpose of disclosure.

### BRIEF DESCRIPTIONS OF THE DRAWINGS

So that the matter in which the above-recited features, advantages and objects of the invention, as well as others which will become clear, are attained and can be understood in detail, more particular descriptions of the invention briefly summarized above may be had by reference to certain embodiments thereof which are illustrated in the appended drawings. These drawings form a part of the specification. It is to be noted, however, that the appended drawings illustrate preferred embodiments of the invention and therefore are not to be considered limiting in their scope.

FIGS. 1A-1C depict the sequences of ERK1 and ERK2 and putative inhibitor binding sites on ERK2 and represent approximate orthogonal views of the protein. FIG. 1A shows the sequence alignment between ERK1 and ERK2 demonstrating 88.2% identity. The sequences comprising the CD and ED domains are boxed and the amino acids differing from ERK2 are underlined. FIG. 1B shows the structure of the phosphorylated form of ERK2 with residues implicated in substrate interactions (yellow with residue number in black). Spheres demarcating putative binding pockets are shown in red for the S1 site, green for the S2 site, and white for the remaining sites (S3-S9). FIG. 1C shows the activation site residues Thr183 and Tyr185 (olive).

FIGS. 2A-2B depict superimposed structures of the unphosphorylated (green) and phosphorylated (purple) forms of ERK2. FIG. 2A illustrates the superimposed ribbon image showing the location and conformational changes associated with the ATP binding domain, activation site, and the ED and CD domains. FIG. 2B illustrates the superimposed ribbon image in the vicinity of the CD (Asp 316 and 319) and ED (Thr 157 and 158) domains.

FIGS. 3A-3B show the molecular weight distribution of top compounds. FIG. 3A shows the molecular weight distributions of the top 20,000 compounds screened against unphosphorylated ERK2 based on normalized and unnormalized vdW attractive energies obtained during primary database screening. Distribution for the entire database is also shown. FIG. 3B shows the molecular weight distributions of the top 500 compounds based on normalized and unnormalized total interaction energies obtained during secondary screening.

FIGS. 4A-4B depict the structures of compounds tested in ERK substrate phosphorylation assays. Compounds 17, 36, 67, 68, 76, 79, 80, and 81 were identified using the unphosphorylated ERK2 protein structure (FIG. 4A). Compounds 86-98 were identified using the phosphorylated ERK2 protein structure (FIG. 4B).

FIGS. 5A-5E demonstrate the effects of test compounds on ERK substrate phosphorylation. HeLa cells were pretreated with or without 100 μM of test compounds for 5 min (FIGS. 5A-5B) or 15 min (FIGS. 5C-5E) prior to the addition of epidermal growth factor (EGF or E) to stimulate the ERK pathway or anisomycin (A) to stimulate p38 MAP kinase. In FIGS. 5A-5B HeLa cells were treated with EGF for 5 minutes. FIG. 5A shows an immunoblot of RSK-1 phosphorylated on Thr573 (pRSK-1). The far left lane is the control (−) with no EGF. The corresponding densitometry graph shows the relative pRSK-1 expression. The control (C) is EGF only treatment. In FIG. 5B cells pretreated with increasing concentrations of compound 76 were stimulated with EGF. ELK-1 phosphorylation on Ser383 (pELK-1) was measured by immunoblotting. The expression of dually phosphorylated ERK1/2 (ppERK1/2) and a-tubulin as a loading control are also shown. FIG. 5C shows immunoblots of Elk-1 phosphorylated on S383 (pElk-1), a-tubulin, and active ERK1/2 (ppERK1/2). The lower graph shows the quantification of the ratio of pElk-1 to a-tubulin as measured by densitometry. The pElk-1 phosphorylation in the presence of the test compounds was compared to the EGF only treatment, which was set at 100%. FIG. 5D shows immunoblots of Rsk-1 phosphorylated on T573 (pRsk-1) and a-tubulin as a loading control. The lower graph shows the quantification of the ratio of pRsk-1 to a-tubulin as measured by densitometry. The pRsk-1 phosphorylation in the presence of the test compounds was compared to the EGF only treatment, which was set at 100%. FIG. 5E shows immunoblot analysis of phosphorylated ATF2 (pATF2) and a-tubulin as a loading control. The lower graph shows the quantification of the ratio pATF2 to a-tubulin as measured by densitometry. The pATF2 phosphorylation in the presence of the test compounds was compared to the anisomycin (A) only treatment, which was set at 100%. The data were reproduced in at least 3 independent experiments.

FIGS. 6A-6B demonstrate the effect of test compounds on ERK2 fluorescence. The percentage of ERK2 fluorescence (F) was plotted against the log concentration in moles/liter (Log [M]) of each test compound using the peak fluorescence in the absence of test compound set at 100% as the reference. Fluorescence titration of ERK2 was done with compounds 36, 67, 76, and 81 and compounds 17, 76 and 79-80 (FIG. 6A) identified using the unphosphorylated ERK2 protein structure. Fluorescence titrations were done using compounds 92-95 and compounds 86, 89 and 98 (FIG. 6B) identified using the phosphorylated ERK2 protein structure.

FIGS. 7A-7E demonstrate inhibition of cell proliferation with test compounds. HeLa, A549, or SUM-159 cells were plated at a low density of ˜200-400 cells per well in the absence or presence of putative ERK docking domain inhibitors. Cell colonies were stained with crystal violet and counted after 6-10 days. In FIG. 7A cells were grown on 10 cm plates in the absence (−) or presence of 100 mM of compound 67, 36, 68, 81, or 76. Colony formation dose response in the presence of the indicated concentrations of compound 76 or 81 for Hela cells (100 mM) (FIG. 7B), A549 cells (50 mM) (FIG. 7C), or SUM-159 cells (50 mM) (FIG. 7D). In FIG. 7E cells were grown on 1.5 cm wells in the absence (−) or presence of of 0, 25, or 75 mM of compound 92, 94, or 95.

FIGS. 8A-8B show predicted binding of active compounds to ERK2. The binding mode of 17 (FIG. 8A) or 76 (FIG. 8B) is shown. The ERK2 structure is shown in gray. The space-filling model of the docked compounds is predicted to form contacts with several amino acids within the groove between Asp316 and Asp319 of the CD domain (blue spheres) and Thr157 and Thr158 of the ED domain (green spheres). Sulfur, oxygen, or nitrogen atoms on the active compounds are indicated as yellow, red, or blue spheres, respectively.

### DETAILED DESCRIPTION OF THE INVENTION

I. Definitions

The use of the word “a” or “an” when used in conjunction with the term “comprising” in the claims and/or the specification may mean “one,” but it is also consistent with the meaning of “one or more,” “at least one,” and “one or more than one.” Some embodiments of the invention may consist of or consist essentially of one or more elements, method steps, and/or methods of the invention. It is contemplated that any method or composition described herein can be implemented with respect to any other method or composition described herein.

The use of the term “or” in the claims is used to mean “and/or” unless explicitly indicated to refer to alternatives only or the alternatives are mutually exclusive, although the disclosure supports a definition that refers to only alternatives and “and/or.” As used herein, the term “antagonist” refers to a biological or chemical agent that acts within the body to reduce the physiological activity of another chemical or biological substance. In the present invention, the antagonist blocks, inhibits, reduces and/or decreases the activity of an extracellular signal-regulated kinase (ERK) of a cell, including, without limitation, a cancer cell. In the present invention, the antagonist combines, binds, associates with an ERK of a cancer cell, such that the ERK is deactivated, meaning reduced biological activity with respect to the biological activity in the diseased state. In certain embodiments, the antagonist combines, binds and/or associates with a docking domain of ERK1, such as a CD, ED, SB or MS docking domain. Alternatively, the antagonist combines, binds and/or associates with a docking domain of ERK2, such as a CD, ED, SB or MS docking domain. The terms antagonist or inhibitor can be used interchangeably herein.

As used herein, the term “compound” is interchangeable with “inhibitor”, “antagonist” or “inhibitory compound” and means a molecular entity of natural, semi-synthetic or synthetic origin that blocks, stops, inhibits, and/or suppresses substrate interactions with a ERK protein docking domain.

As used herein, the term “contacting” refers to any suitable method of bringing one or more of the compounds described herein or other inhibitory agent into contact with an ERK protein, as described, or a cell comprising the same. In vitro or ex vivo this is achieved by exposing the ERK protein or cells comprising the same to the compound or inhibitory agent in a suitable medium. For in vivo applications, any known method of administration is suitable as described herein.

As used herein, the terms “effective amount” or “therapeutically effective amount” are interchangeable and refer to an amount that results in an improvement or remediation of the symptoms of the disease or condition. Those of skill in the art understand that the effective amount may improve the patient's or subject's condition, but may not be a complete cure of the disease and/or condition.

As used herein, the term “inhibit” refers to the ability of the compound to block, partially block, interfere, decrease, reduce or deactivate extracellular signal-regulated kinase (ERK). Thus, one of skill in the art understands that the term inhibit encompasses a complete and/or partial loss of activity of ERK. ERK activity may be inhibited by occlusion or closure of the docking domain, by disruption of the interaction with the substrate, by sequestering ERK and/or the substrate, or by other means. For example, a complete and/or partial loss of activity of the ERK as may be indicated by a reduction in phosphorylation, a reduction in cell proliferation, a reduction in toxicity of a cancer therapy, or the like.

As used herein, the term “heteroatom” or “heterocyclic” refers to an atom in an organic molecule or compound that is nitrogen, oxygen, sulfur, phosphorus or a halogen or an aromatic compound comprising the heteroatom. It is particularly contemplated that a heteroatom is nitrogen, oxygen or sulfur.

As used herein, the term “neoplasm” refers to a mass of tissue or cells characterized by, inter alia, abnormal cell proliferation. The abnormal cell proliferation results in growth of these tissues or cells that exceeds and is uncoordinated with that of the normal tissues or cells and persists in the same excessive manner after the stimuli which evoked the change ceases or is removed. Neoplastic tissues or cells show a lack of structural organization and coordination relative to normal tissues or cells which usually results in a mass of tissues or cells which can be either benign or malignant. As would be apparent to one of ordinary skill in the art, the term “cancer” refers to a malignant neoplasm.

As used herein, the term “treating” or the phrase “treating a cancer” or “treating a neoplasm” includes, but is not limited to, halting the growth of the neoplasm or cancer, killing the neoplasm or cancer, or reducing the size of the neoplasm or cancer. Halting the growth refers to halting any increase in the size or the number of or size of the neoplastic or cancer cells or to halting the division of the neoplasm or the cancer cells. Reducing the size refers to reducing the size of the neoplasm or the cancer or the number of or size of the neoplastic or cancer cells.

As used herein, the term “subject” refers to any target of the treatment.

II. Present Invention

In one embodiment of the present invention there is provided a method of inhibiting an activity of an extracellular signal-regulated kinase (ERK) in a cell, comprising contacting the cell with an inhibitory compound that selectively binds to one or more docking domain regions of the ERK thereby inhibiting an ERK activity associated with an ERK substrate binding thereto. In all aspects of this embodiment the extracellular signal-recognition kinase may be ERK1 or ERK2. Also, in all aspects the docking domain region comprises one or more of a CD domain, an ED domains, a SB domain, or a MS domain. Representative examples of the inhibitory compound are compound 17, compound 36, compound 76, compound 79, compounds 80-81, or compounds 92-95. In an aspect of this embodiment the cell is a neoplastic cell. Examples of a neoplastic cell are those cells comprising a breast cancer, a lung cancer, a cervical cancer, a pancreatic cancer, a bladder cancer, a colon cancer, or a cancer having a Ras mutation.

In another embodiment of the present invention there is provided a method inhibiting proliferation of a neoplastic cell, comprising contacting the neoplastic cell with an inhibitory compound that selectively inhibits binding of a substrate of an extracellular signal-regulated kinase to one or more docking domain regions thereof whereby proliferation of the neoplastic cell is inhibited; wherein said inhibitory compound is compound 17, compound 76, compound 89, compound 92, compound 93, or compound 95. In all aspects of this embodiment, the extracellular signal-recognition kinases, the docking domains and the cancers are as described supra. In yet another embodiment of the present invention there is provided a method of treating a cancer in a subject, comprising administering an inhibitory compound that selectively binds to one or more docking domain regions of an extracellular signal-recognition kinase to reduce proliferation of cells comprising the cancer upon binding said inhibitory compound thereto, thereby treating the cancer in the subject.

Further to this embodiment the method may comprise administering an anticancer drug to the subject. In aspects of this embodiment, the anticancer drug may be administered concurrently or sequentially with the inhibitory compound. In another aspect of this embodiment a dosage of the anticancer drug is lower than a dosage required when the anticancer drug is administered singly, thereby reducing toxicity of the anticancer drug to the individual. Examples of anticancer drugs are cisplatin, oxaliplatin, carboplatin, doxorubicin, a camptothecin, paclitaxel, methotrexate, vinblastine, etoposide, docetaxel hydroxyurea, celecoxib, fluorouracil, busulfan, imatinib mesylate, alembuzumab, aldesleukin, and cyclophosphamide.

The inhibitory compounds may be compound 17, compound 36, compound 76, compound 79, compound 80, compound 81, or one of compounds 86-98. Preferably, the inhibitory compounds may be compound 17, compound 76, compound 86, compound 89, compound 92, compound 93, compound 94, or compound 95. Additionally, in all aspects of these embodiments the extracellular signal-recognition kinases, the docking domains and the cancers are as described supra.

In a related embodiment the present invention provides a method of reducing toxicity of a cancer therapy in an individual in need thereof, comprising administering to the individual an inhibitory compound that selectively binds to one or more docking domain regions of an extracellular signal-recognition kinase (ERK) and an anticancer drug, where a dosage of the anticancer drug administered with the inhibitory compound is lower than a dosage required when the anticancer drug is administered singly, thereby reducing toxicity of the cancer therapy to the individual. In aspects of this embodiment, the anticancer drug may be administered concurrently or sequentially with the inhibitory compound. In all aspects the extracellular signal-recognition kinases, the docking domains, the inhibitory compounds, the anticancer drugs and the cancers are as described supra.

In still another embodiment of the present invention there is provided a method of identifying an inhibitor of substrate binding to a docking domain region of an extracellular signal-reduction kinase (ERK), comprising designing a test compound that binds to one or more docking domain regions in ERK, but does not interfere with the ATP binding domain, wherein the design is based at least in part on computer-aided drug design (CADD) modeling; measuring the level of phosphorylation of an ERK substrate protein in the presence or absence of the test compound; and comparing the level of protein phosphorylation in the presence of the test compound with the level of protein phosphorylation in the absence of the test compound, wherein a decrease in protein phosphorylation in the presence of the test compound is indicative that the test compound is an inhibitor of binding to the docking domain region in ERK.

Further to this embodiment the method comprises screening the inhibitor for anti-cell proliferative activity directed against neoplastic cells. In this further embodiment screening comprises contacting a culture of the neoplastic cells having an activated ERK activity with the inhibitor; and comparing the amount of cell proliferation of the neoplastic cells in the presence of the inhibitor with the amount of cell proliferation of the neoplastic cells in the -absence of the inhibitor, where a decrease in cell proliferation in the presence of the inhibitor compared to cell proliferation in the absence of the inhibitor is indicative that the inhibitory compound has the ability to prevent cell proliferation in neoplastic cells.

In all aspects of these embodiments the extracellular signal-recognition kinase may be ERK1 or ERK2. Also, in all aspects the docking domain region comprises one or more of a CD domain, an ED domains, a SB domain, or a MS domain. Further to these aspects the ERK inhibitor forms a bond with the CD domain, and more specifically with Asp316, Asp319 or a combination thereof of same. Alternatively, the ERK inhibitor forms a bond with the ED domain, and more specifically, with at least one of residues Glu79, Asn80,Gln130, Arg133, Tyr314, Gln313 of same. Again in all aspects the neoplastic cells and cancers are as described supra.

In a related embodiment there is provided an inhibitory compound identified by the methods of screening for an inhibitor of substrate binding to a docking domain region of an extracellular signal-reduction kinase (ERK) and of inhibiting cell proliferation of a neoplastic cell. In another related embodiment there is provided an ERK inhibitory compound having a chemical structure comprising one or more substituted or unsubstituted heterocyclic aromatic ring moieties covalently coupled in a size and shape designed to bind to one or more docking domain regions of an extracellular signal-reduction kinase without interfering with an ATP binding domain therein, said design based at least in part on computer-aided drug design models.

In all aspects of this embodiment the heterocyclic aromatic ring comprises a nitrogen, sulfur, or oxygen or a combination thereof. In a particular aspect the substituted heterocyclic aromatic ring comprises at least one of a pendant heteroatom, a pendant moiety having one or more heteroatoms, a side-chain having one or more heteroatoms or a combination thereof Additionally, in all aspects the extracellular signal-reduction kinase is ERK1 or ERK2 and the docking domain region comprises one or more of a CD domain, an ED domains, a SB domain, or a MS domain. Further to these aspects the ERK inhibitor forms a bond with the CD domain, and more specifically with Asp316, Asp319 or a combination thereof of the same. Alternatively, the ERK inhibitor forms a bond with the ED domain, and more specifically, with at least one of residues Glu79, Asn80,Gln130, Arg133, Tyr314, Gln313 of the same.

In a related embodiment there is provided an ERK inhibitory compound having a chemical structure comprising one or more substituted or unsubstituted heterocyclic aromatic ring moieties comprise nitrogen, sulfur, or oxygen heteroatoms or a combination thereof and further comprises at least one of a pendant heteroatom, a pendant moiety having one or more heteroatoms, a side-chain having one or more heteroatoms or a combination thereof covalently coupled in a size and shape, said substituted heterocyclic aromatic ring moieties designed to bind to one or more docking domain regions of an extracellular signal-reduction kinase without interfering with an ATP binding domain therein. In this embodiment the docking domain regions and the amino acid residues to which the ERK inhibitory compounds forms bonds are as described supra.

In a further related embodiment there is provided an ERK inhibitory compound having a chemical structure comprising one or more substituted or unsubstituted heterocyclic aromatic rings comprising a nitrogen, sulfur, or oxygen or a combination, and said substituted heterocyclic aromatic ring comprises at least one of a pendant heteroatom, a pendant moiety having one or more heteroatoms, a side-chain having one or more heteroatoms or a combination thereof covalently coupled in a size and shape designed to bind within a CD or ED docking domain region of an extracellular signal-reduction kinase without interfering with an ATP binding domain therein, wherein said ERK inhibitory compound forms a bond with Asp316, Asp319 or a combination thereof comprising the CD domain. Alternatively, the ERK inhibitor forms a bond with the ED domain, and more specifically, with at least one of residues Glu79, Asn80,Gln130, Arg133, Tyr314, Gln313 of the same.

Provided herein are compounds that inhibit ERK protein docking domains by selectively blocking substrate interactions and methods of using these compounds to treat pathophysiological conditions having an unregulated cell proliferative component. By targeting unique regions on ERK, increased selectivity of these compounds in blocking ERK-specific phosphorylation of RSK-1 and ELK-1 may be achieved compared to typical kinase inhibitors that act as competitive inhibitors of ATP. The ERK proteins may target dozens of different substrates in vivo. Selective inhibition of substrates involved in unregulated cell proliferation may be achieved by targeting ERK docking domains. Computer-aided drug design (CADD) provides for the identification of compounds that disrupt ERK interactions with substrates involved in pathophysiological conditions while preserving ERK interactions with substrates needed for normal metabolic processes and cell maintenance.

An effective amount of an ERK inhibitor that may be administered to a cell includes a dose of about 0.0001 nM to about 2000 μM. More specifically, doses of an agonist to be administered are from about 0.01 nM to about 2000 μM; about 0.01 μM to about 0.05 μM; about 0.05 μM to about 1.0 μM; about 1.0 μM to about 1.5 μM; about 1.5 μM to about 2.0 μM; about 2.0 μM to about 3.0 μM; about 3.0 μM to about 4.0 μM; about 4.0 μM to about 5.0 μM; about 5.0 μM to about 10 μM; about 10 μM to about 50 μM; about 50 μM to about 100 μM; about 100 μM to about 200 μM; about 200 μM to about 300 μM; about 300 μM to about 500 μM; about 500 μM to about 1000 μM; about 1000 μM to about 1500 μM and about 1500 μM to about 2000 μM. Of course, all of these amounts are exemplary, and any amount in-between these points is also expected to be of use in the invention.

The ERK inhibitor or related-compound (derivative) thereof can be administered parenterally or alimentary. Parenteral administrations include, but are not limited to intravenously, intradermally, intramuscularly, intraarterially, intrathecally, subcutaneous, or intraperitoneally U.S. Pat. Nos. 6,613,308, 5,466,468, 5,543,158; 5,641,515; and 5,399,363 (each specifically incorporated herein by reference in its entirety). Alimentary administrations include, but are not limited to orally, buccally, rectally, or sublingually.

The administration of the therapeutic compounds and/or the therapies of the present invention may include systemic, local and/or regional administrations, for example, topically (dermally, transdermally), via catheters, implantable pumps, etc. Alternatively, other routes of administration are also contemplated such as, for example, arterial perfusion, intracavitary, intraperitoneal, intrapleural, intraventricular and/or intrathecal. The skilled artisan is aware of determining the appropriate administration route using standard methods and procedures. Other routes of administration are discussed elsewhere in the specification and are incorporated herein by reference.

Treatment methods will involve treating an individual with an effective amount of a composition containing ERK inhibitor or related-compound thereof. An effective amount is described, generally, as that amount sufficient to detectably and repeatedly to ameliorate, reduce, minimize or limit the extent of a disease or its symptoms. More specifically, it is envisioned that the treatment with the ERK inhibitor or related-compounds thereof will inhibit ERK protein docking of a protein substrate, wherein the protein substrate would have been phosphorylated by the ERK if not for the inhibition, will inhibit cell proliferation of a cancer cell, specifically a neoplastic cell, and/or in embodiments in which a cancer drug is present, wherein the cancer drug is applied before, during or after the ERK inhibitor, will reduce the toxicity of said cancer drug evidenced by a reduced dosage of the cancer drug if applied in combination with the ERK inhibitor is necessary to achieve the same therapeutic benefit as compared to the control dosage applied in the absence of the ERK inhibitor.

The effective amount of ERK inhibitor or related-compounds thereof to be used are those amounts effective to produce beneficial results, particularly with respect to stroke treatment, in the recipient animal or patient. Such amounts may be initially determined by reviewing the published literature, by conducting in vitro tests or by conducting metabolic studies in healthy experimental animals. Before use in a clinical setting, it may be beneficial to conduct confirmatory studies in an animal model, preferably a widely accepted animal model of the particular disease to be treated. Preferred animal models for use in certain embodiments are rodent models, which are preferred because they are economical to use and, particularly, because the results gained are widely accepted as predictive of clinical value.

As is well known in the art, a specific dose level of active compounds such as ERK inhibitor or related-compounds thereof for any particular patient depends upon a variety of factors including the activity of the specific compound employed, the age, body weight, general health, sex, diet, time of administration, route of administration, rate of excretion, drug combination, and the severity of the particular disease undergoing therapy. The person responsible for administration will determine the appropriate dose for the individual subject. Moreover, for human administration, preparations should meet sterility, pyrogenicity, general safety and purity standards as required by FDA Office of Biologics standards.

One of skill in the art realizes that the effective amount of the ERK inhibitor or related-compound thereof can be the amount that is required to achieve the desired result: reduction/inhibition in phosphorylation, reduction/inhibition of cell proliferation, reduction of toxicity of a cancer drug, etc.

Administration of the therapeutic ERK inhibitor composition of the present invention to a patient or subject will follow general protocols for the administration of therapies used in cancer treatment taking into account the toxicity, if any, of the ERK inhibitor and/or, in embodiments of combination therapy, the toxicity of the cancer drug. It is expected that the treatment cycles would be repeated as necessary. It also is contemplated that various standard therapies, as well as surgical intervention, may be applied in combination with the described therapy.

Pharmaceutical Formulations and Methods of Treating

Compositions of the Present Invention

The present invention also contemplates therapeutic methods employing compositions comprising the active substances disclosed herein. Preferably, these compositions include pharmaceutical compositions comprising a therapeutically effective amount of one or more of the active compounds or substances along with a pharmaceutically acceptable carrier.

As used herein, the term “pharmaceutically acceptable” carrier means a non-toxic, inert solid, semi-solid liquid filler, diluent, encapsulating material, formulation auxiliary of any type, or simply a sterile aqueous medium, such as saline. Some examples of the materials that can serve as pharmaceutically acceptable carriers are sugars, such as lactose, glucose and sucrose, starches such as corn starch and potato starch, cellulose and its derivatives such as sodium carboxymethyl cellulose, ethyl cellulose and cellulose acetate; powdered tragacanth; malt, gelatin, talc; excipients such as cocoa butter and suppository waxes; oils such as peanut oil, cottonseed oil, safflower oil, sesame oil, olive oil, corn oil and soybean oil; glycols, such as propylene glycol, polyols such as glycerin, sorbitol, mannitol and polyethylene glycol; esters such as ethyl oleate and ethyl laurate, agar; buffering agents such as magnesium hydroxide and aluminum hydroxide; alginic acid; pyrogen-free water; isotonic saline, Ringer's solution; ethyl alcohol and phosphate buffer solutions, as well as other non-toxic compatible substances used in pharmaceutical formulations.

Wetting agents, emulsifiers and lubricants such as sodium lauryl sulfate and magnesium stearate, as well as coloring agents, releasing agents, coating agents, sweetening, flavoring and perfuming agents, preservatives and antioxidants can also be present in the composition, according to the judgment of the formulator. Examples of pharmaceutically acceptable antioxidants include, but are not limited to, water soluble antioxidants such as ascorbic acid, cysteine hydrochloride, sodium bisulfite, sodium metabisulfite, sodium sulfite, and the like; oil soluble antioxidants, such as ascorbyl palmitate, butylated hydroxyanisole (BHA), butylated hydroxytoluene (BHT), lecithin, propyl gallate, aloha-tocopherol and the like; and the metal chelating agents such as citric acid, ethylenediamine tetraacetic acid (EDTA), sorbitol, tartaric acid, phosphoric acid and the like.

Dose Determinations

By a “therapeutically effective amount” or simply “effective amount” of an active compound, such as compound 17, compound 36, compound 76, compound 79, compound 80, compound 81, or one of compounds 86-98 , is meant a sufficient amount of the compound to treat a cancer at a reasonable benefit/risk ratio applicable to any medical treatment. It will be understood, however, that the total daily usage of the active compounds and compositions of the present invention will be decided by the attending physician within the scope of sound medical judgment. The specific therapeutically effective dose level for any particular patient will depend upon a variety of factors including the disorder, disease or injury being treated and the severity of the disorder, disease or injury; activity of the specific compound employed; the specific composition employed; the age, body weight, general health, sex and diet of the patient; the time of administration, route of administration, and rate of excretion of the specific compound employed; the duration of the treatment; drugs used in combination or coinciding with the specific compound employed; and like factors well known in the medical arts.

Toxicity and therapeutic efficacy of such compounds can be determined by standard pharmaceutical procedures in cell assays or experimental animals, e.g., for determining the LD50 (the dose lethal to 50% of the population) and the ED50 (the dose therapeutically effective in 50% of the population). The dose ratio between toxic and therapeutic effects is the therapeutic index and it can be expressed as the ratio LD50/ED50. Compounds which exhibit large therapeutic indices are preferred. While compounds that exhibit toxic side effects may be used, care should be taken to design a delivery system that targets such compounds to the site of affected tissue in order to minimize potential damage to uninfected cells and, thereby, reduce side effects.

The data obtained from the cell culture assays and animal studies can be used in formulating a range of dosage for use in humans. The dosage of such compounds lies preferably within a range of circulating concentrations that include the ED50 with little or no toxicity. The dosage may vary within this range depending upon the dosage form employed and the route of administration utilized. For any compound used in the method of the invention, the therapeutically effective dose can be estimated initially from cell based assays. A dose may be formulated in animal models to achieve a circulating plasma concentration range that includes the IC50 (ie., the concentration of the test compound which achieves a half-maximal inhibition of symptoms) as determined in cell culture. Such information can be used to more accurately determine useful doses in humans. Levels in plasma may be measured, for example, by high performance liquid chromatography.

The total daily dose of the active compounds of the present invention administered to a subject in single or in divided .doses can be in amounts, for example, from 0.01 to 25 mg/kg body weight or more usually from 0.1 to 15 mg/kg body weight. Single dose compositions may contain such amounts or submultiples thereof to make up the daily dose. In general, treatment regimens according to the present invention comprise administration to a human or other mammal in need of such treatment from about 1 mg to about 1000 mg of the active substance(s) of this invention per day in multiple doses or in a single dose of from 1 mg, 5 mg, 10 mg, 100 mg, 500 mg or 1000 mg.

In certain situations, it may be important to maintain a fairly high dose of the active agent in the blood stream of the patient, particularly early in the treatment. Such a fairly high dose may include a dose that is several times less than its use in other indications. For example, as per the IPCSINTOX databank, the usual dose regimen of cisplatin when given as a single agent is 50-120 mg/M² by intravenous infusion once every 3 to 4 weeks or 15-20 mg/m² by intravenous infusion daily for five consecutive days every 3 to 4 weeks. For example, in one embodiment of the present invention directed to a method of inhibiting proliferation of a neoplastic cell of a subject by administering to the subject a formulation containing an effective amount of a compound that blocks ERK and a pharmaceutically acceptable carrier; such formulations may contain from about 0.1 to about 100 grams of ERK inhibitor or from about 0.5 to about 150 milligrams of the ERK inhibitors of the present invention.

In certain situations of treating a cancer, it may be important to maintain a fairly high dose of the active agent to ensure delivery to the desired site of the patient, particularly early in the treatment. Hence, at least initially, it may be important to keep the dose relatively high and/or at a substantially constant level for a given period of time, preferably, at least about six or more hours, more preferably, at least about twelve or more hours and, most preferably, at least about twenty-four or more hours.

Formulations and Administration

The compounds of the present invention may be administered alone or in combination or in concurrent therapy with other agents which affect the targeted cell(s). Liquid dosage forms for oral administration may include pharmaceutically acceptable emulsions, microemulsions, solutions, suspensions, syrups and elixirs containing inert diluents commonly used in the art, such as water, isotonic solutions, or saline. Such compositions may also comprise adjuvants, such as wetting agents; emulsifying and suspending agents; sweetening, flavoring and perfuming agents.

Injectable preparations, for example, sterile injectable aqueous or oleaginous suspensions may be formulated according to the known art using suitable dispersing or wetting agents and suspending agents. The sterile injectable preparation may also be a sterile injectable solution, suspension or emulsion in a nontoxic parenterally acceptable diluent or solvent, for example, as a solution in 1,3-butanediol. Among the acceptable vehicles and solvents that may be employed are water, Ringer's solution, U.S.P. and isotonic sodium chloride solution. In addition, sterile, fixed oils are conventionally employed as a solvent or suspending medium. For this purpose any bland fixed oil can be employed including synthetic mono- or diglycerides. In addition, fatty acids such as oleic acid are used in the preparation of injectables. The injectable formulation can be sterilized, for example, by filtration through a bacteria-retaining filter, or by incorporating sterilizing agents in the form of sterile solid compositions, which can be dissolved or dispersed in sterile water or other sterile injectable medium just prior to use.

In order to prolong the effect of a drug, it is often desirable to slow the absorption of a drug from subcutaneous or intramuscular injection. The most common way to accomplish this is to inject a suspension of crystalline or amorphous material with poor water solubility. The rate of absorption of the drug becomes dependent on the rate of dissolution of the drug, which is, in turn, dependent on the physical state of the drug, for example, the crystal size and the crystalline form. Another approach to delaying absorption of a drug is to administer the drug as a solution or suspension in oil. Injectable depot forms can also be made by forming microcapsule matrices of drugs and biodegradable polymers, such as polylactide-polyglycoside. Depending on the ratio of drug to polymer and the composition of the polymer, the rate of drug release can be controlled. Examples of other biodegradable polymers include polyorthoesters and polyanhydrides. The depot injectables can also be made by entrapping the drug in liposomes or microemulsions, which are compatible with body tissues.

Suppositories for rectal administration of the drug can be prepared by mixing the drug with a suitable non-irritating excipient, such as cocoa butter and polyethylene glycol which are solid at ordinary temperature but liquid at the rectal temperature and will, therefore, melt in the rectum and release the drug.

Solid dosage forms for oral administration may include capsules, tablets, pills, powders, gelcaps and granules. In such solid dosage forms the active compound may be admixed with at least one inert diluent such as sucrose, lactose or starch. Such dosage forms may also comprise, as is normal practice, additional substances other than inert diluents, e.g., tableting lubricants and other tableting aids such as magnesium stearate and microcrystalline cellulose. In the case of capsules, tablets and pills, the dosage forms may also comprise buffering agents. Tablets and pills can additionally be prepared with enteric coatings and other release-controlling coatings. Solid compositions of a similar type may also be employed as fillers in soft and hard-filled gelatin capsules using such excipients as lactose or milk sugar as well as high molecular weight polyethylene glycols and the like.

The active compounds can also be in micro-encapsulated form with one or more excipients as noted above. The solid dosage forms of tablets, capsules, pills, and granules can be prepared with coatings and shells such as enteric coatings and other coatings well known in the pharmaceutical formulating art. They may optionally contain opacifying agents and can also be of a composition that they release the active ingredient(s) only, or preferably, in a certain part of the intestinal tract, optionally in a delayed manner. Examples of embedding compositions which can be used include polymeric substances and waxes.

Dosage forms for topical or transdermal administration of a compound of this invention further include ointments, pastes, creams, lotions, gels, powders, solutions, sprays, inhalants or patches. Transdermal patches have the added advantage of providing controlled delivery of active compound to the body. Such dosage forms can be made by dissolving or dispersing the compound in the proper medium. Absorption enhancers can also be used to increase the flux of the compound across the skin. The rate can be controlled by either providing a rate controlling membrane or by dispersing the compound in a polymer matrix or gel. The ointments, pastes, creams and gels may contain, in addition to an active compound of this invention, excipients such as animal and vegetable fats, oils, waxes, paraffins, starch, tragacanth, cellulose derivatives, polyethylene glycols, silicones, bentonites, silicic acid, talc and zinc oxide, or mixtures thereof.

The method of the present invention employs the compounds identified herein for both in vitro and in vivo applications. For in vivo applications, the invention compounds can be incorporated into a pharmaceutically acceptable formulation for administration. Those of skill in the art can readily determine suitable dosage levels when the invention compounds are so used. As employed herein, the phrase “suitable dosage levels” refers to levels of compound sufficient to provide circulating concentrations high enough to effectively block a ERK docking domain and reduce phosphorylation and/or cell proliferation in vivo.

In accordance with a particular embodiment of the present invention, compositions comprising at least one ERK antagonist compound (as described above), and a pharmaceutically acceptable carrier are contemplated. Exemplary pharmaceutically acceptable carriers include carriers suitable for oral, intravenous, subcutaneous, intramuscular, intracutaneous, and the like administration. Administration in the form of creams, lotions, tablets, dispersible powders, granules, syrups, elixirs, sterile aqueous or non-aqueous solutions, suspensions or emulsions, and the like, is contemplated.

For the preparation of oral liquids, suitable carriers include emulsions, solutions, suspensions, syrups, and the like, optionally containing additives such as wetting agents, emulsifying and suspending agents, sweetening, flavoring and perfuming agents, and the like.

For the preparation of fluids for parenteral administration, suitable carriers include sterile aqueous or non-aqueous solutions, suspensions, or emulsions. Examples of non-aqueous solvents or vehicles are propylene glycol, polyethylene glycol, vegetable oils, such as olive oil and corn oil, gelatin, and injectable organic esters such as ethyl oleate. Such dosage forms may also contain adjuvants such as preserving, wetting, emulsifying, and dispersing agents. They may be sterilized, for example, by filtration through a bacteria-retaining filter, by incorporating sterilizing agents into the compositions, by irradiating the compositions, or by heating the compositions. They can also be manufactured in the form of sterile water, or some other sterile injectable medium immediately before use. The active compound is admixed under sterile conditions with a pharmaceutically acceptable carrier and any needed preservatives or buffers as may be required.

The treatments may include various “unit doses.” Unit dose is defined as containing a predetermined quantity of the therapeutic composition calculated to produce the desired responses in association with its administration, e.g., the appropriate route and treatment regimen. The quantity to be administered, and the particular route and formulation, are within the skill of those in the clinical arts. Also of import is the subject to be treated, in particular, the state of the subject and the protection desired. A unit dose need not be administered as a single injection but may comprise continuous infusion over a set period of time.

Combination Treatments

In the context of the present invention, it is contemplated that an ERK antagonist (ERK inhibitor) or derivative thereof may be used in combination with an additional therapeutic agent to more effectively treat a cancer, and/or decrease cancer cell proliferation. In some embodiments, it is contemplated that a conventional therapy or agent, including but not limited to, a pharmacological therapeutic agent may be combined with the antagonist or related-compound of the present invention.

Pharmacological therapeutic agents and methods of administration, dosages, etc. are well known to those of skill in the art (see for example, the “Physicians Desk Reference”, Goodman & Gilman's “The Pharmacological Basis of Therapeutics”, “Remington's Pharmaceutical Sciences”, and “The Merck Index, Eleventh Edition”, incorporated herein by reference in relevant parts), and may be combined with the invention in light of the disclosures herein. Some variation in dosage will necessarily occur depending on the condition of the subject being treated. The person responsible for administration will, in any event, determine the appropriate dose for the individual subject, and such individual determinations are within the skill of those of ordinary skill in the art.

Non-limiting examples of a pharmacological therapeutic agent that may be used in the present invention include an antihyperlipoproteinemic agent, an antiarteriosclerotic agent, an anticholesterol agent, an antiinflammatory agent, an antithrombotic/fibrinolytic agent, anticoagulant, antiplatelet, vasodilator, and/or diuretics. Thromoblytics that are used can include, but are not limited to prourokinase, streptokinase, and tissue plasminogen activator (tPA) Anticholesterol agents include but are not limited to HMG-CoA Reductase inhibitors, cholesterol absorption inhibitors, bile acid sequestrants, nicotinic acid and derivatives thereof, fibric acid and derivatives thereof. HMG-CoA Reductase inhibitors include statins, for example, but not limited to atorvastatin calcium (Lipitor®), cerivastatin sodium (Baycol®), fluvastatin sodium (Lescol®), lovastatin (Advicor(®), pravastatin sodium (Pravachol®), and simvastatin (Zocor®).

Agents known to reduce the absorption of ingested cholesterol include, for example, Zetia®. Bile acid sequestrants include, but are not limited to cholestryramine, cholestipol and colesevalam. Other anticholesterol agents include fibric acids and derivatives thereof (e.g., gemfibrozil, fenofibrate and clofibrate); nicotinic acids and derivatives thereof (e.g., nician, lovastatin) and agents that extend the release of nicotinic acid, for example niaspan.

Antiinflammatory agents include, but are not limited to non-sterodial anti-inflammatory agents, e.g., naproxen, ibuprofen, and celeoxib, and sterodial anti-inflammatory agents, e.g., glucocorticoids. Anticoagulants include, but are not limited to heparin, warfarin, and coumadin. Antiplatelets include, but are not limited to aspirin, and aspirin related-compounds, for example acetaminophen. Diuretics include, but are not limited to such as furosemide (Lasix®), bumetanide (Bumex®), torsemide (Demadex®), thiazide & thiazide-like diuretics, e.g., chlorothiazide (Diuril®) and hydrochlorothiazide (Esidrix®), benzthiazide, cyclothiazide, indapamide, chlorthalidone, bendroflumethizide, metolazone), amiloride, triamterene, and spironolacton. Vasodilators include, but are not limited to nitroglycerin,

Thus, in certain embodiments, the present invention comprises co-administration of an ERK inhibitor with a cancer drug. Co-administration of these two compounds may decrease the therapeutic effective amount of the cancer drug.

When an additional therapeutic agent, as long as the dose of the additional therapeutic agent does not exceed previously quoted toxicity levels, the effective amounts of the additional therapeutic agent may simply be defined as that amount effective to inhibit a ERK docking domain from interaction with an intended protein substrate, thereby inhibiting phosphorylation of that intended substrate, when administered to an animal. This may be easily determined by monitoring the animal or patient and measuring those physical and biochemical parameters of health and disease that are indicative of the success of a given treatment. Such methods are routine in animal testing and clinical practice. This may be achieved by contacting the cell with a single composition or pharmacological formulation that includes both agents, or by contacting the cell with two distinct compositions or formulations, at the same time, wherein one composition includes an ERK inhibitor or derivatives thereof and the other includes the additional agent.

Alternatively, treatment with ERK inhibitor or related-compounds thereof may precede or follow the additional agent treatment by intervals ranging from minutes to hours to weeks to months. In embodiments where the additional agent is applied separately to the cell, one would generally ensure that a significant period of time did not expire between the time of each delivery, such that the agent would still be able to exert an advantageously combined effect on the cell. In such instances, it is contemplated that one would contact the cell with both modalities within about 1-24 hr of each other and, more preferably, within about 6-12 hr of each other.

Inhibitors of cell proliferation of cancer cells may be natural, semi-synthetic or synthetic compounds that have been designed or screened from chemical libraries or may be a synthetic derivative or analog compound having a structure similar to a known inhibitor. Inhibitors of ERK substrate docking identified by the methods described herein can block proliferation of cancer cells without affecting normal cell proliferation. Such inhibitors may be used to inhibit proliferation of neoplastic cells, to treat a cancer or to reduce the toxicity of a cancer drug to normal cells. Nonlimiting examples of cancer drugs contemplated in the present invention include cisplatin, oxaliplatin, carboplatin, doxorubicin, a camptothecin, paclitaxel, methotrexate, vinblastine, etoposide, docetaxel hydroxyurea, celecoxib, fluorouracil, busulfan, imatinib mesylate, alembuzumab, aldesleukin, and cyclophosphamide.

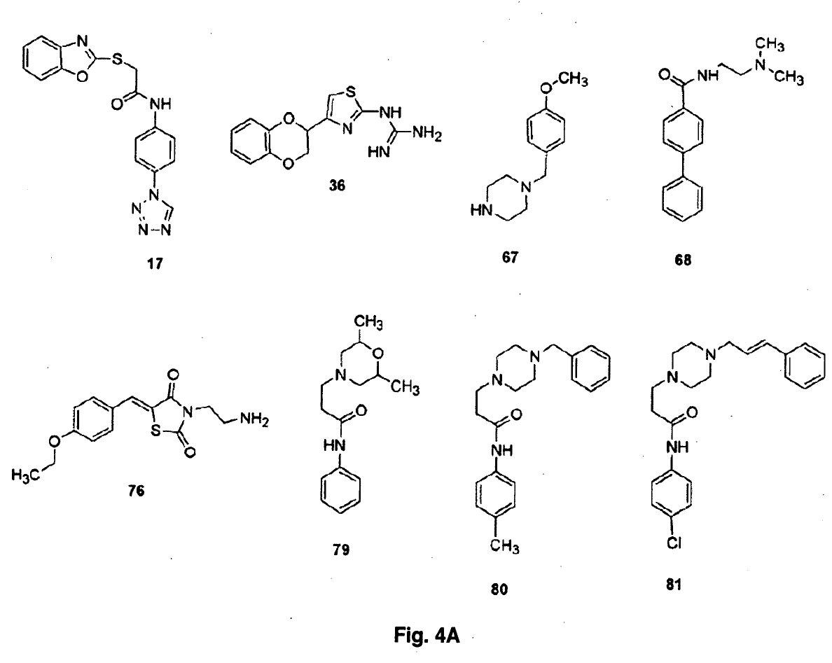

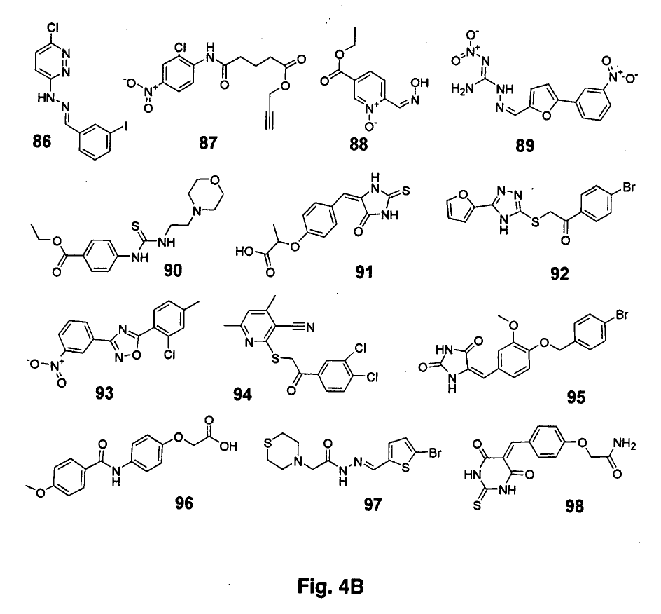

Accordingly, using the phosphorylated or unphosphorylated ERK2 crystal structure in a CADD (22) screening of a virtual database, small molecular weight compounds that disrupt ERK function by interacting with binding sites of one or more docking domain regions of ERK2 to selectively inhibit ERK-specific phosphorylation of substrates have been identified. Moreover, biological assays revealed that these lead compounds were effective in preventing proliferation of cancer cell lines. The inhibitory compounds so identified using unphosphorylated ERK2 include compound 17, compound 36, compound 76, compound 79, and compounds 80-81. The inhibitory compounds so identified using phosphorylated ERK2 include compounds 86-98. Preferably, compounds 17, 76, 89, and 92-95 are useful as therapeutics. The structures are shown in FIGS. 4A-4B.

Potential inhibitory compounds identified by CADD modeling may be screened for inhibitory activity directed against substrate binding to ERK docking domain regions, for example CD, ED, SB, or MS. Without being limiting, for example, an inhibitory compound may inhibit substrate binding to the CD and ED docking domain region of ERK2. The inhibitory compound may block, stop, inhibit, and/or suppress substrate binding to one or more of these docking regions at one or more binding sites S1-S9 (see Table 2).

For example, ERK-associated phosphorylation activity may be assayed in the presence of ATP and a substrate phosphorylated via ERK and in the presence or absence of the potential inhibitor. A decrease in substrate phosphorylation in the presence of the potential inhibitor compared to substrate phosphorylation in the absence of the potential inhibitor is indicative that it has an ability to inhibit ERK substrate binding within the docking domain region of ERK. Such enzyme assays are known and standard in the art.

Subsequently, any inhibitor of the MLK-associated activity may be used in a cell proliferation assay. For example, a cancer cell culture having activated ERK activity is contacted with a potential inhibitory compound. A decrease in cell proliferation, as compared to control, may be determined by standard assays, such as a colony formation assay, trypan blue exclusion or other such assay known in the art.

It is contemplated that a test compound can include derivatives thereof (referred to interchangeably as ‘derivative compound’) which may or may not be designed using the CADD modeling described herein. Predicted binding orientations of derivative compounds may be verified using X-ray crystallography or NMR spectroscopy, as is known in the art. Additionally, the CADD screen may be expanded to identify additional molecules that can act as lead compounds for the development of novel ERK inhibitors that can be used for experimental and clinical purposes.

For example the inhibitors may be synthetic compounds designed to have a chemical structure that at least includes one or more heterocyclic aromatic rings in the structure. An aromatic ring moiety is covalently coupled to be a size and shape to bind within the docking domain region of ERK without interfering with or inhibiting ATP binding to ERK. A heteroatom comprising the heterocyclic ring may be one or more of nitrogen, sulfur, oxygen or a combination thereof. The aromatic ring moiety may be substituted or unsubstituted. Substituent atoms or molecules may be, but are not limited to, one or more of a pendant heteroatom, a pendant moiety having one or more heteroatoms, a side-chain having one or more heteroatoms or a combination thereof. The chemical structure is sufficient to form one or more ionic bonds and/or one or more pi (□) bonds with amino acid residues from one or more of the CD, ED, SB, or MS domains. For example, an inhibitor may form a bond with an Asp316, Asp319 or a combination thereof comprising the CD domain and/or with at least one of Glu79, Asn80, Gln130, Arg133, Tyr314, Gln313 comprising the ED domain. Generally, Table 2 in Example 2 provides a list of substrates and putative ERK2 docking domain sites with available residues.

The inhibitory compounds provided herein may be used to treat any subject, preferably a mammal, more preferably a human, having a pathophysiological condition characterized by the presence of transformed cells, e.g., a neoplasm, such as, but not limited, to a cancer. For example, a cancer may be a breast cancer, a lung cancer, a cervical cancer, a pancreatic cancer, a bladder cancer, a colon cancer, or another cancer having a Ras mutation. Administration of the inhibitory compound to a subject results in growth arrest of cancer cells without affecting the growth of a normal cell. Thus, cell proliferation is inhibited and a therapeutic effect, up to and including killing the cancer, is achieved thereby treating the cancer. It is contemplated that the compounds of the present invention may be used to inhibit proliferation of non-malignant neoplastic diseases and disorders.

Such an approach of selective inhibition of ERK substrates may also reduce toxicity to normal cells, which is observed with many of the current chemotherapies. An anticancer drug may be administered concurrently or sequentially with the compounds of the present invention. The effect of co-administration with an effective compound is to lower the dosage of the anticancer drug normally required that is known to have at least a minimal pharmacological or therapeutic effect against a cancer or cancer cell, for example, the dosage required to eliminate a cancer cell. Concomitantly, toxicity of the anticancer drug to normal cells, tissues and organs is reduced without reducing, ameliorating, eliminating or otherwise interfering with any cytotoxic, cytostatic, apoptotic or other killing or inhibitory therapeutic effect of the drug on the cancer cells.

The compounds and anticancer drugs can be administered independently, either systemically or locally, by any method standard in the art, for example, subcutaneously, intravenously, parenterally, intraperitoneally, intradermally, intramuscularly, topically, enterally, rectally, nasally, buccally, vaginally or by inhalation spray, by drug pump or contained within transdermal patch or an implant. Dosage formulations of these compounds and of the anti-cancer drugs may comprise conventional non-toxic, physiologically or pharmaceutically acceptable carriers or vehicles suitable for the method of administration.

The compounds and anticancer drugs or pharmaceutical compositions thereof may be administered independently one or more times to achieve, maintain or improve upon a therapeutic effect. It is well within the skill of an artisan to determine dosage or whether a suitable dosage of either or both of the inhibitory compound and anticancer drug comprises a single administered dose or multiple administered doses. An appropriate dosage depends on the subject's health, the progression or remission of the cancer, the route of administration and the formulation used.

The following example(s) are given for the purpose of illustrating various embodiments of the invention and are not meant to limit the present invention in any fashion.

### EXAMPLE 1

Cells and Reagents

HeLa (human cervical carcinoma), A549 (human lung carcinoma), HT1080 (human fibrosarcoma), or MDA-MB-468 (breast adenocarcinoma) cell lines were purchased from American Type Culture Collection (ATCC, Manassas, Va.). The estrogen receptor negative breast cancer cells, SUM-159, were obtained from the University of Michigan Human Breast Cancer Cell SUM-Lines. All cell lines were cultured in a complete medium consisting of Dulbecco's modified Eagle medium (DMEM) supplemented with 10% fetal bovine serum (FBS) and antibiotics (Penicillin, 100 U/ml; Streptomycin, 100 μg/ml) (Invitrogen, Carlsbad, Calif.). Epidermal growth factor (EGF) and phorbol 12-myristate 13-acetate (PMA) were purchased from Sigma (St. Louis, Mo.) and used at final concentrations of 25 ng/ml and 0.1 μM, respectively. Antibodies against phosphorylated Rsk-1 (pT573), Elk-1 (pS383), and ERK (pT183, pY185) were purchased from Cell Signaling Technologies (Woburn, Mass.), Santa Cruz Biotech. (Santa Cruz, Calif.), and Sigma, respectively. The α-tubulin antibody was purchased from Sigma.

ERK Substrate Phosphorylation and Immunoblotting

Prior to harvesting, cells were pre-incubated with the test compounds for 15-20 minutes and then stimulated with EGF or anisomycin to activate the ERK or p38 MAP kinase pathways, respectively. Control and treated cells were washed twice with cold phosphate buffered saline (PBS, pH 7.2; Invitrogen) and proteins were collected following cell lysis with 300 μl of cold tissue lysis buffer (20 mM Tris, pH 7.4, 137 mM NaCl, 2 mM EDTA, 1% Triton X-100, 0.1% SDS, 25 mM β-glycerophosphate, 2 mM sodium pyrophosphate, 10% glycerol, 1 mM sodium orthovanadate, 1 mM phenylmethylsulfonyl fluoride (PMSF), 1 mM benzamidine), allowed to incubate on ice for about 10 minutes and then centrifuged at 20,000 (X g) to clarify the lysates of insoluble material. The lysates then were diluted with an equal volume of 2X SDS-sample buffer and the proteins were separated on SDS-PAGE for immunoblot analysis. Immunoblot analysis was done as previously described (41-43).

To expedite analysis of large numbers of samples from a diverse number of cell lines, protein lysates from control and treated cells are spotted onto nitrocellulose membrane using a Minifold-1 spot blot (96 well) apparatus (Whatman/Schleicher and Schuell). The nitrocellulose is sectioned into four quadrants each containing 24 spots. Each sample is spotted within each of these quadrants, which are cut and are immunoblotted with a specific antibody. Initially the four sections of membrane are immunoblotted using antibodies against pELK-1, pRSK-1, ppERK1/2, and a-tubulin. This method of analysis only works with antibodies that have been shown to be specific for the protein of interest after SDS-PAGE and immunoblotting. The four antibodies mentioned fit these criteria. Quantification of the immunoblots is done by densitometry (44). In addition, conditions, such as protein loading amounts and exposure times are established so that quantification is within the linear range of the densitometer.

An antibody microarray approach (45) is used to analyze the phosphorylation status of multiple substrates under control and treated conditions. This technology is current available through several vendors (eg. BD Biosciences). Proteins extracted from control and treated cells are labeled with fluorescent dyes (Cy3 and Cy5). The labeled proteins are then incubated with the antibody microarray containing a customized assortment of phosphorylation-specific antibodies against ERK-specific substrates. Table 1 lists some of the available phospho-specific antibodies against ERK substrates that are tested. The validation of antibody specificity first is done by SDS-PAGE and immunoblotting. In addition, the effects of substrates specific for the other major MAP kinases, JNK, and p38, are tested.

TABLE 1

| | Phosphorylation | |
|---|---|---|
| | sites | Company |
| ERK | | |
| RSK-1 | T359/S360/T573 | Cell Signaling |
| RSK-3 | T353/356 | ″ |
| ELK-1 | S383 | ″ |
| c-Myc | T58/S62 | ″ |
| MNK-1 | T197/T202 | ″ |
| PPAR-g | S112 | Chemicon |
| Tyrosine hydroxylase S31 | | ″ |
| Connexin-43 | S255 | Santa Cruz |
| Estrogen receptor-a | S118 | ″ |
| Tau | S199/S202 | Santa Cruz/BioSource |
| JNK | | |
| c-Jun | S63/S73 | Cell Signaling |
| p53 | T81 | ″ |
| p38 | | |
| ATF-2 | T71 | Cell Signaling |
| MAPKAPK-2 | T334 | ″ |
| MNK-1 | T197/T202 | ″ |
| Stat-1 | S727 | ″ |
| MSK-1 | S369/S376 | ″ |

Cell Proliferation Assays

Trypsinized cells were plated on 12 or 24 well plates at low densities (200-400 cells per well) in the absence (DMSO only) or presence of test compounds. In additional experiments, cells were first allowed to adhere to the culture dishes for 16 hours prior to treatment with test compounds. The treated cells received a single dose of the test compounds at the beginning of the experiments. The control and treated cells were grown for 8-14 days to allow the formation of colonies. Cells then were fixed for 10 minutes in 4% paraformaldehyde and stained with 0.2% crystal violet (in 20% methanol) for 1-2 minutes. Following several washes with distilled water, the colonies (containing at least 40 cells) were counted. Each individual experiment was repeated on at least 3 separate occasions.

Protein Purification

ERK2 was purified as described previously (46) with some modifications. Briefly, (His)ₓ₆-tagged ERK2 was expressed in bacteria and the cells were harvested in BugBuster protein extraction reagent (EMD Biosciences, San Diego, Calif.). Clarified lysates were loaded onto a Talon Co²⁺-IMAC affinity chromatography resin column (BD Biosciences, San Jose, Calif.) and the bound protein was eluted using increasing concentrations of imidazole. SDS-PAGE electrophoresis and Coomassie blue staining were used to identify the eluted fractions containing the ERK2 protein. The ERK2 protein concentration was determined using Bradford Reagent (Sigma). Phosphorylated ERK2 is generated by dual phosphorylation on the Thr183 and Tyr185 active sites by incubation with a constitutively active MEK1 mutant as previously described (46).

Fluorescence Titrations

Direct binding interactions between ERK2 and the biologically active compounds is determined using fluorescence spectroscopy (47). Experiments measure the changes in the intrinsic ERK2 fluorescence due to the presence of aromatic amino acids, with the indole group of tryptophan being the major fluorophore with absorption and emission maxima around 280 and 340 nanometers (nm), respectively. Fluorescence spectra were recorded with a Luminescence Spectrometer LS50 (Perkin Elmer, Boston, Mass.). For all experiments, ERK2 protein was diluted into 20 mM Tris-HCl, pH 7.5. Titrations were performed by increasing the test compound concentration while maintaining the ERK2 protein concentration at 3 μM.

Unphosphorylated and phosphorylated ERK2 typically are incubated with 1, 5, 10, 25, 50, 75, or 100 mM of the biologically active compounds and the fluorescence intensity is measured. If necessary, higher inhibitor concentrations are used to saturate fluorescence quenching. The excitation wavelength was 295 nm and fluorescence was monitored from 300 to 500 nm. All reported fluorescence intensities are relative values and are not corrected for wavelength variations in detector response. Dissociation constants, K_{D}, were determined using reciprocal plots, 1/ν vs 1/[I], where ν represents the percent occupied sites calculated assuming fluorescence quenching to be directly proportion to the percentage of occupied binding sites, [I] represents the concentration of the inhibitor compound and the slope of the curve equals the K_{D} (48-49). Because the test compounds contain aromatic structures, the emission spectra of the active compounds in the absence of ERK2 is be determined. Based on the fluorescence of the active compounds in the absence of ERK2, the ERK2 fluorescence intensity changes are corrected for compound fluorescence as required.

X-Ray Crystallography

The unphosphorylated (His)₆-tagged ERK2 is expressed and purified as described above with additional purification through Mono Q and Phenyl Superose columns as previously described (50). Briefly, the purified protein is dialyzed against storage buffer (25 mM Tris-HCl [pH 7.4], 100 mM NaCl, 1 mM EDTA, and 0.1 mM DTT). Prior to crystallization, a 3-fold molar excess of the test compound is added to ERK2 (8 mg/ml) in storage buffer for 24 hr at 4° C. Crystals are grown in hanging drops at 16° C. by mixing 1 ml protein/peptide solution with 1 ml well solution containing 20% PEG 8000, 0.1 M sodium cacodylate, pH 7.0, and 0.2 M calcium acetate, and identified in Crystal Screen I (Hampton Research). Structure determination is done as previously reported (27,51).

Pharmacokinetic Analysis

Tissue and plasma area under the curve from 0 to 24 hours (AUCO-₀₋₂₄) are determined using Bailer's method (52). This method permits calculation of the variance associated with the AUC, thus yielding a 95 % confidence interval (95% CI). Equation 1 is used to calculate the AUC,

$$( {AUC}_{j} ) = {\sum }_{q = 1}^{m} {c}_{q} {\overline{y}}_{j , q} mn \qquad ( 3 )$$

where cq=½ D2 for q=1, ½ (Dq+Dq+1) for q=2 to q=m−1, cq=½ Dm for q=m; j is the number of groups, D is the time interval, m is the number of time points, and q is any given time point from 1 to m. y_{j,q} is the sample mean of the response at time q in group j. In this case number of groups j=1. The variance associated with the AUC was calculated using equation 2,

$${s}^{2} ( {AUC}_{j} ) = {\sum }_{q = 1}^{m} {c}_{q}^{2} [ \frac{{s}_{jq}^{2}}{{n}_{jq}} ] \qquad ( 2 )$$

where s²_{jq} is the variance associated with the response for each group at time point q, and n_{jq} is the number of animals per group at time point q. Clearance is estimated for the Bailer calculated AUC by using equation 3.

$$Cl = \frac{Dose}{AUC} \qquad ( 1 )$$

The maximum concentration (Cₘₐₓ) and time of maximum concentration were the observed values. The drug exposure and pharmacokinetic parameters of maximum concentration (Cₘₐₓ), time of maximum concentration (tₘₐₓ), area under the concentration versus time curve (AUC), and terminal half-life (t_{1/2}) are calculated compared between the treatment drugs.

### EXAMPLE 2

ERK2 Substrates and Putative Docking Domain Sites in Unphosphorylated ERK2

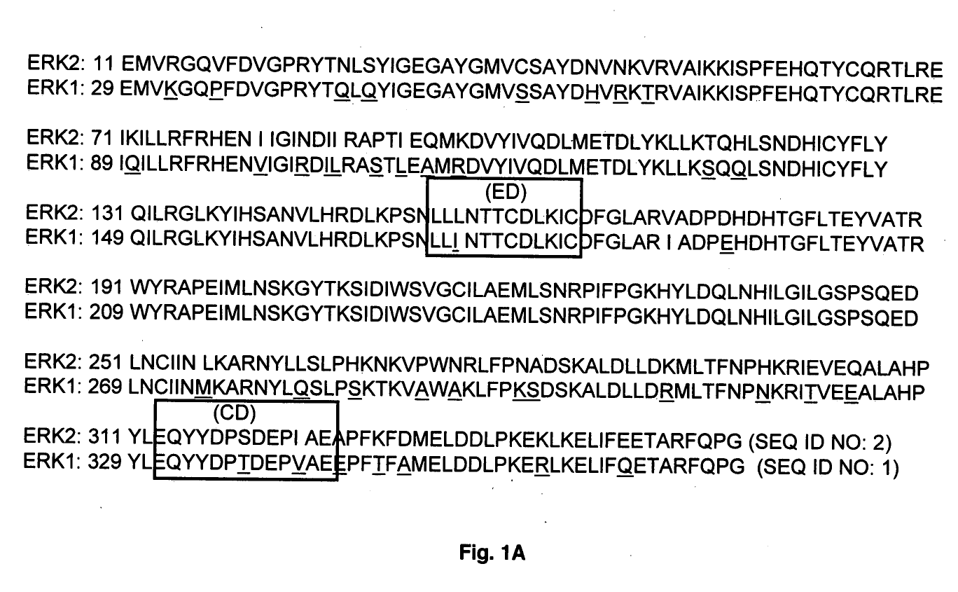

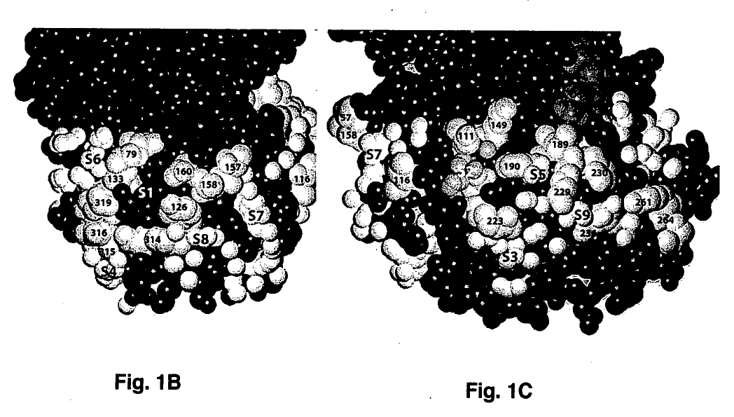

FIG. 1A shows the sequence alignment between ERK1 (SEQ ID NO: 1) and ERK2 (SEQ ID NO: 2) demonstrating 88.2% identity and the location of the CD and ED domains. FIGS. 1B-1C show the residues that have been identified as being involved in ERK2-substrate interactions (14,34). As shown, a large number of residues may be involved in substrate interactions and these residues are distributed over a large region of the C-terminal portion of the protein. To identify novel putative binding sites in the vicinity of the substrate-binding residues, the program SPHGEN was used to identify concave regions on the entire protein surface and fill them with virtual spheres. Clusters of these spheres are used to direct the placement of ligands during virtual database screening as in Examples 3 and 4. Of the identified clusters, those with 5 or more spheres and with one or more spheres within 5 Å of any of the substrate-binding residues were identified and are shown as red, green, or white vdW spheres. Putative binding pockets, as defined by the sphere clusters, are identified as S1-S3.

Table 2 presents a summary of experimental data on ERK2 substrates and the ERK2 residues that interact with those substrates along with the associated putative binding sites that are shown in FIGS 1B-1C. S1 originally was selected as it is adjacent to the common domain (CD) known to be important for the binding of a number of substrates and to the ED domain implicated in MEK1/2 and ELK-1 specific binding. The remaining sites were identified based on the density of the spheres in the clusters and their location relative to the residues of interest. It is contemplated that the ERK2 residues involved in MKP3 and MEK1 interactions also may be involved in regulating the efficiency of ERK interactions with other substrates.

TABLE 2

| | | | Putative | |
|---|---|---|---|---|
| | Site | | Binding | |
| Substrate | Name | Residues | Sites | Refs |
| Nonspecific | CD | Asp316, Asp319 | S1, S6 | (34) |
| Binding | | | | |
| MEK1 binding | ED | Thr157, Thr158 | S1, S7 | (14, 34) |
| ELK-1 | ED | Thr157, Thr158 | S1, S7 | (14, 34) |
| MKP3 binding | CD | Glu79, Tyr126, Arg133 | S1, S6, S8 | (14, 16) |
| | | Asp160, Tyr314, | | |
| | | Asp316, Asp319 | | |
| MKP3 activation | SB | Tyr111, Thr116, Leu119 | S2, S3, S5 | (14) |
| | | Lys149, Arg189, Trp190 | | |
| | | Glu218, Arg223, Lys229 | | |
| | | His230 | | |
| MEK1 binding | CD | Tyr315, Asp316, | S1, S6, S4 | (14) |
| | | Asp319, Asp320, | | |
| MEK1 binding | MS | His230, Asn236 | S9 | (53) |
| | | Tyr261, Ser264 | | |

S1: Between CD and ED domains, may impact MEK1/2 interactions and ELK-1, but lack of ERK substrate specificity is possible. S2: Between residues 111, 149, 190, 218, and 223; indicated to effect MKP3 activation. S3: Below 223; possible specificity for MKP3 activation; location on edge of identified residues may facilitate specificity. S4: Close to 315/316 implicated in MEK1 activation and binding, although 316 also implicated in MKP3 binding; location on edge of identified residues may facilitate specificity. S5: Between 189, 190, 223, 229, and 230 all involved in MKP3 activation. S6: In vicinity of 79, 133, 316, and 319 that are implicated for binding of a variety of substrates, may be general ERK substrate inhibitor. S7: Below 157/158 related to MEK1 and ELK-1 specificity; extended binding groove with decreased probability of having nonspecific effects associated with CD residues. S8: Close to 126 and 314 implicated in MKP3 activation; location on edge of identified residues may facilitate specificity. S9: Between 230 and 236 implicated in MEK1 specificity.
Comparison of Unphosphorylated and Phosphorylated ERK2 Conformation

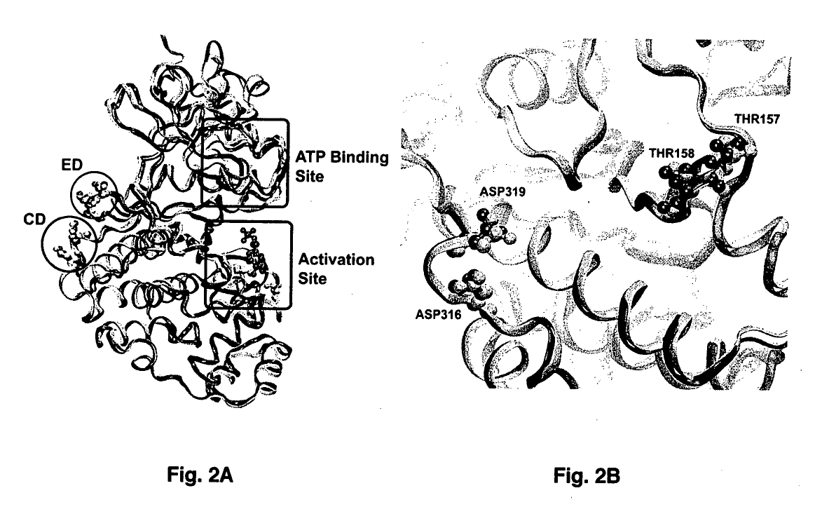

Comparison of the conformations of the two forms of the protein from crystallographic studies (27-28) show both the overall structures (FIG. 2A) as well as the regions in the vicinity of the CD and ED domains to be similar (FIG. 2B). The considerable overlap is consistent with the reported lack of a conformational change in the ED region of active ERK as measured by changes in deuterium exchange (54) However, some changes in deuterium exchange rates within the region containing the CD domain have been observed (54). Such differences, which may be due to either subtle differences in structure or changes in the flexibility of the protein, indicate that targeting active ERK2 may identify additional ERK docking domain inhibitors.

### EXAMPLE 3

General CADD Method for Compound Screening Database Searching

The 3D structures of ERK2 in both the unphosphorylated and phosphorylated states (28,50) are available from the Protein DataBank (29). Charges and hydrogens are added to the proteins using SYBYL6.4 (Tripos, Inc.). All database searching calculations are carried out with DOCK 4.0.1, that includes in-house modifications, using flexible ligands based on the anchored search method (31). Ligand-protein interaction energies are approximated by the sum of the electrostatic and van der Waals (vdW, steric) components as calculated by the GRID method (35,55) implemented in DOCK using default values. In the GRID model a 3D lattice of hypothetical points is overlaid on the protein and the electrostatic and vdW potential due to the protein at each point is calculated. Interaction energies of ligands are then calculated based on the potential grid, rather than directly with the protein, yielding a significant saving in computer resources. The grid extends 15 Å beyond the respective sphere sets used for initial ligand placement in all dimensions, insuring that the docked compounds are totally encompassed by the grid.

Identification of binding sites in the ERK2 docking domain is performed using the sphere sets calculated with the DOCK associated program SPHGEN. The solvent accessible surface (32) is calculated with the program DMS (33) using a surface density of 2.76 surface points per A² and a probe radius of 1.4 Å² following which the spheres are generated for the entire protein via SPHGEN. From the full sphere set, all sphere clusters with one or more spheres within 5 Å of any of the non-hydrogen atoms of residues experimentally identified to contribute to substrate binding are saved, as shown and discussed in Table 2 above.

Final selection of the putative binding sites for full docking are performed as follows. Each sphere cluster is analyzed individually, with individual spheres not part of the central region of the cluster manually deleted, thereby focusing the cluster. Preliminary docking is then performed against each cluster on 10,000 compounds, from which the binding response is calculated. The binding response is a modified scoring term that accounts for the spatial overlap of each docked compound with the sphere set such that if there is no overlap the binding response is 0 and if the overlap is ideal the value is 1, with the binding response for a particular binding site obtained by averaging over all 10,000 compounds. Visually, if the binding response is low, the docked compounds are spread over a wide area around the binding site while in the case of a site with a binding response approaching one the compounds are docked in a focused fashion overlaying the binding site. The binding response of each of the sites in Table 2 above are calculated with those sites with higher binding responses being prioritized.

Primary database searching is performed using the phosphorylated ERK2 3D structure on a 3D chemical database of over 3 million compounds. This includes commercially available compounds and compounds in the NCI 3D chemical database (56). The database has been compiled and converted from 2D structures to 3D structures (26,57).

Initiation of the database searches involves selection of compounds that contain 10 or less rotatable bonds and between 10 and 40 non-hydrogen atoms. Ligand flexibility is considered by dividing each compound into a collection of non-overlapping rigid segments, e.g. rings, referred to as anchors. Each anchor then is docked separately into the binding site in 200 different orientations, based on different overlap of the anchor atoms with the sphere set, and energy minimized. The remainder of each molecule is built onto the anchor in a stepwise fashion until the entire molecule is built, with each step corresponding to a rotatable bond. At each step the dihedral about the rotatable bond, which is connecting the new segment being added to the previously constructed portion of the molecule, is sampled in 10° increments and the lowest energy conformation is selected based on the interaction energy. During the build-up procedure selected conformers are removed based on energetic considerations and maximization of diversity of the conformations being sampled (37-38). The orientation of the compound with the most favorable interaction energy is finally selected.

From the initial DOCK runs, the top 50,000 compounds are selected based on normalized vdW attractive interactive energies. Use of the vdW attractive energy, versus total energy or electrostatic energy, forces the procedure to select compounds with structures that sterically complement the binding site. If electrostatics were included in the selection, compounds that did not fit the binding site well, but had strong favorable electrostatic interactions, i.e. ion pairs, would be chosen. The normalization procedure is designed to control the molecular weight (MW) of the selected compounds (46). Use of N^{1/2} normalization where N is the number of non-hydrogen atoms in the compounds, typically selects compounds with an average molecular weight of 320 daltons. Such compounds are smaller than the average molecular weight of pharmaceutically active compounds based on the World Drug index. The smaller molecular weight of the lead compounds allows the addition of functional groups during lead optimization efforts (58).

Secondary database searching of the top 50,000 compounds from each binding site is performed by applying a more rigorous secondary docking approach, termed method 2, which includes simultaneous energy minimization of the anchor during the iterative build-up procedure. In addition, method 2 docking is performed against both the phosphorylated and unphosphorylated ERK2 structures for each of the 50,000 compounds. The inclusion of two structures at this stage of docking partially accounts for the lack of receptor binding site flexibility during the database search. For each compound the most favorable score from the two ERK2 protein conformations is used for the final ranking. Scoring is based on the total interaction energy, as compounds dominated by electrostatic energies would have been eliminated during method 1 screening. Normalization is used again for selection of the desired molecular weight distribution.

From this procedure the top 1000 compounds are selected for chemical similarity clustering. In chemical similarity clustering, each compound is assigned a “fingerprint” based on the types of atoms in the compound and the connectivity between those atoms (e.g. atoms bonded to each other, atoms bonded to one of the atoms in the first bonded pair, and so on). The fingerprints of different compounds are then used to cluster the compounds into structurally similar sets based on the Tanimoto Similarity Index (39). This process yields approximately 100 clusters. One or two compounds are selected from each cluster for biological assay. This final selection process considers stability, potential toxicity, and solubility, where solubilities are estimated via calculated log P values using the Molecular Operating Environment (MOE, Chemical Computing Group). Selected compounds may be purchased from the appropriate vendor.

Lead Validation

For an active compound to be considered a viable lead for additional studies it is ideal if it can be shown that the compound is a member of a class of active compounds. This may be performed by identifying compounds that are chemically similar to the active compounds based on the fingerprint analysis. Such an approach is similar to pharmacophore searching where it has been shown that compounds with similar structures should have similar biological activities (59). Application of this approach is necessary as the initial database search emphasizes chemical diversity during compound selection. In addition, with compounds that are active, but at decreased levels, identifying and assaying structurally similar compounds can identify compounds with enhanced activity, essentially rescuing low activity compounds and validating them as leads. It is contemplated that obtaining experimental data for collection of structurally similar compounds provides a basis for systematic structure-activity studies required for lead optimization.

Similarity searches targeting the 3 million compound database are performed as described. In these searches, the Tanimoto coefficient is adjusted to identify approximately 50 compounds for each active compound which are obtained for biological assay. These searches are performed following removal of extraneous substituents, e.g. methyl, amine or acid moieties, from the compounds that do not participate in linkers between ring systems. For molecules that contain three or more ring systems, similarity searches are done on analogs that contain only two rings. This approach allows for a wider variety of structurally similar compounds to be identified.

Alternative Methods for DOCK Based Database Searching

DOCK based database searching makes a number of simplifications in order to minimize computer requirements, allowing for the databases of 3 million compounds to be searched. Of these simplifications the two most important are 1) the lack of conformational flexibility in the protein and 2) the simplified scoring function. If either of these assumptions becomes problematic, the following steps can be taken.

The assumption of a rigid protein during the docking procedure is necessary due to the large number of degrees of freedom in proteins, e.g., a conservative estimate is 10^{N}, where N is the number of amino acids. Two conformations of the ERK2 protein based on the crystal structures are used in the method 2 search. If this number of conformations is deemed inadequate, additional conformations can be generated via molecular dynamics (MD) simulations of ERK2 in aqueous solution, using the molecular modeling program CHARMM (60-61). Molecular dynamics simulations are performed to sample the conformational space of the putative binding sites described above. These additional conformations, typically 5, are included in the method 2 search in addition to the crystal structures.

Alternate scoring methods are attempted if significant improvements in the hit rates are not obtained. One alternate approach that may be applied with both method 1 and method 2 searches is consensus scoring (62-63). In this approach, several scoring functions are applied simultaneously, yielding improved estimation of the relative rankings of the docked compounds. This includes knowledge-based or potential of mean force (PMF) scoring methods that have been shown to yield improvements in the selection of correct orientations of ligands and have the advantage that they implicitly include certain aspects of solvation effects (64-65). Alternate approaches that may be used if deemed appropriate include generalized linear response methods (66-67) and free energy of solvation based on the Generalized Born (GB) model (68), including models included in the CHARMM program (69-70).

### EXAMPLE 4

Identification of Inhibitors of ERK2 S1 binding site in CD and ED CADD in silico Primary Screening using Unphosphorylated ERK2

The ERK2 structure (FIGS. 2A-2B) is bilobal in nature and is typical of many kinases where the amino and carboxyl lobes are separated by a hinge region (27). Upon phosphorylation of Thr183 and Tyr185 a conformational change brings the N-terminal lobe containing the ATP binding site in proximity to the C-terminal lobe to allow phosphate transfer onto substrate proteins. It has been suggested that substrate proteins interactions with ERK2 are determined by a common docking (CD) and ED domain regions in the C-terminus that interact with substrate binding motifs (14,34). This region was selected for the identification of putative binding sites, as inhibitors that bind to such sites will have the potential of blocking ERK2 substrate-protein interactions, with the inhibition potentially being specific for certain substrate proteins.

Sphere sets were calculated and sphere clusters in the region of the CD and ED docking domains in ERK2, which are important for interactions with the protein substrates, were identified. Based on mutagenesis experiments, residues involved in intermolecular interactions were used to select the docking site. These include Asp316 and Asp319 in the C-terminus (16), which are part of the common docking (CD) domain, and residues Thr157 and Thr158, which contribute to the ED docking domain (34). Spheres within both 10 Å of the CD domain and 12 Å of the ED domain were selected. The resulting sphere set contained 11 spheres and was located in the groove or cleft between the CD and ED domains as shown in FIG. 2A. The GRID box dimensions were 25.3×26.6×27.3 Å³ centered around the sphere set to ensure that docked molecules were within the grid. The compounds that were screened had between 10 and 40 heavy atoms and less than 10 rotatable bonds.

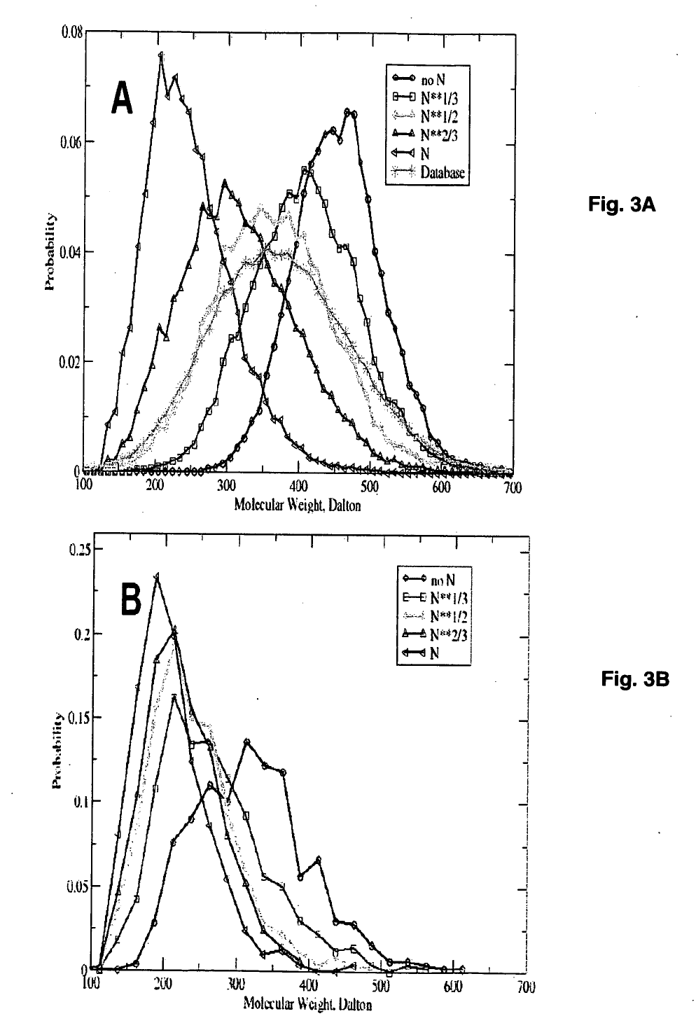

Use of the vdW attractive energy without any normalization yielded an average molecular weight for the top scoring compounds of 457 Da. This means that approximately half of those compounds are above a MW of 500 Da. As drug-like compounds typically have molecular weights below 500 Da (40) and the lead compounds have even lower molecular weights (71), it is desirable to select compounds with lower molecular weights via the normalization procedure. Using N, N^{2/3}, N^{1/2}, and N^{1/3} normalization the average molecular weights were 248, 317, 368, and 410 Da, respectively. FIG. 3A shows how larger powers of N shift the molecular weight distribution towards lower molecular weight values. To select the normalization procedure for compound selection it should be noted that the molecular weight probability distribution of the entire database screened in this Example is centered at 364 Da. Thus, N normalization was chosen since lead compounds of lower molecular weight are desired.

It should be noted that significant overlap of compounds occurs for the different normalization schemes. Of 20,000 compounds selected via N normalization, 11,355 compounds were common in the N^{2/3}set, 6540 in the N^{1/2}set, 3292 in the N^{1/3} set and 815 were in the set of non-normalized compounds. Thus, it may be assumed that compounds with highly favorable interaction orientations with the protein binding site are not being excluded by the normalization procedure.

CADD in silico Secondary Screening using Unphosphorylated ERK2

After the primary screening, compounds were chosen for the secondary screening based on their normalized vdW attractive interaction energy scores. Compound selection based on the DOCK energy score favors compounds with higher molecular weight since their size contributes to the energy score. To minimize this size bias, an efficient procedure by which the DOCK energies are normalized by the number of heavy atoms N or by a power of N was applied as in Equation 4 (36):
IE_{norm,vdW}=IE_{vdW}/N^{x} (4).
Normalization of the vdW energies was done with x, which is a factor empirically selected to correct for the bias of IE-based scoring methods to favor large molecular-weight compounds. The molecular weight distributions of the top 20,000 compounds in each category were compared to the molecular weight of the database.

The total interaction energies of the top 20,000 compounds obtained in this Example were normalized and the molecular weight distributions of the top 500 compounds in each set using different powers of N were determined (FIG. 3B). For the top 500 compounds selected via the N, N^{2/3}, N^{1/2}, and N^{1/3} normalization, the average distributions were 210, 226, 238, and 267 Da, respectively. The average for the top 500 compounds without normalizing the energies was 321 Da. The top 500 scoring compounds in the set obtained after N^{1/3} normalization was chosen to avoid molecules which were too small, thereby lacking adequate structure diversity for lead or drug-like candidates.

Compounds 17, 36, 67-68, 76, and 79-81 selected via CADD were purchased from ChemDiv (San Diego, Calif.) or ChemBridge (San Diego, Calif.) and dissolved in DMSO at a stock concentration of 25, 50, or 100 mM. The purity of the active compounds was verified by mass spectrometry and thin-layer chromatography using 90% chloroform and 10% methanol as the solvent.

CADD in silico Secondary Screening using Phosphorylated ERK2

This methodology, while successful, does not include the flexibility of the protein during docking. To partially account for this omission, the 20,000 compounds from the primary screen were docked against the 3D structure of the phosphorylated form of ERK2 using the secondary docking approach. Docking was performed by dividing each compound into non-overlapping rigid segments connected by rotatable bonds. Segments with more than 5 heavy atoms were used as anchors, each of which was docked into the binding site in 250 orientations and minimized. The remainder of the molecule was built around the anchor in a stepwise fashion by adding other segments connected through rotatable bonds. At each step, the dihedral of the rotatable bond was sampled in increments of 10° and the lowest energy conformation was selected. All rotatable bonds were minimized simultaneously during the stepwise building of the molecule. Pruning of the conformations ensured conformational diversity and more favorable energies (37-38).

Energy scoring was performed with a distant-dependent dielectric, with a dielectric constant of 4, and using an all atom model. The total interaction energies for the best orientation of each were then normalized using x=0, 0.33, 0.5, 0.67, and 1.0 yielding five sets of 500 compounds from each normalization. Based on analysis of the molecular weight distributions of the five sets the x=0 and x=0.33 sets, with average molecular weights of 264 and 255 a.m.u., respectively, were selected and the two sets combined. This yielded a set of approximately 700 unique compounds for similarity clustering after removing those compounds common to both sets.

Chemical similarity clustering of the ˜700 unique compounds was performed to maximize the chemical diversity of the final compounds selected for biological assay. Clustering calculations were performed using the program MOE (Chemical Computing Group, Inc.). The Jarvis-Patrick algorithm, as implemented in MOE, was used to cluster the compounds using the MACC_BITS fingerprinting scheme and Tanimoto coefficient (TC). It first calculates the MACC_BITS fingerprints which encode the 2D structural features for each compound into linear bit strings of data. The pairwise similarity matrix between each compound was calculated based on the TC values (39). TC is one of the metrics available that provides a similarity score by dividing the fraction of features common to both molecules by the total number of features. The similarity matrix is then converted into a second matrix in which each TC value is replaced by a 0 or 1 representing similarity values below and above the threshold value (S) provided by the user, respectively. The rows of the new matrix are treated as fingerprints and the ‘TC’ value between each is calculated. Molecules with values above the selected overlap threshold (T) are put in the same cluster. A 70-40 similarity/overlap value was used to cluster the compounds.

Compounds for experimental assay were chosen from the individual clusters with emphasis on compounds with drug-like physical characteristics as defined by Lipinski et al. (40). Properties considered were the MW, number of hydrogen donors (NHD) and acceptors (NHA) and the logP values as calculated by MOE. However, exceptions were made when all compounds in a cluster had one or more physical characteristics beyond the defined range (40). In addition, only those compounds that were not previously studied (72) were selected with a majority of those compounds selected from clusters in which there were no compounds that had been previously tested.

From this process a total of 45 novel compounds were selected to test in biological assays. The molecular weights of compounds identified using active ERK2 ranged from 188 to 486 a.m.u. with an average and standard deviation of 388+68 a.m.u. Compounds 86-98 were obtained from commercial vendors and purified as described herein. Five compounds (89 and 92-95) have been shown to be active in ERK substrate phosphorylation assays.

CADD-Screened Compounds

FIGS. 4A-4B show the chemical structures for some of the compounds that have been tested for their ability to inhibit ERK-mediated substrate phosphorylation. These include compounds 17, 36, 67, 68, 76, 79, 80, and 81, which were developed against the CD and ED domain (S1 site) using unphosphorylated ERK2 and compounds 86-98, which were developed against the S1 site using the phosphorylated (active) ERK2 protein structure. All compounds except compounds 36 and 68 showed some inhibition of ERK-mediated phosphorylation of RSK-1. Compound 36 was used as control as it had little effect on ERK substrate phosphorylation. The structure of compound 68 was included because it appeared to enhance ERK phosphorylation of RSK-1.

As shown, the compounds have diverse chemical structures, although some similarities are evident. For example, 17, 79, 80 and 81 have amide moieties directly adjacent to aromatic rings with many of compounds including piperazine groups. The advantage of having chemically diverse structures as this stage of the project is, during future lead optimization efforts, to maximize the potential that one or more of the compounds have the desired bioavailability properties as well as specifically targeting ERK-substrate interactions.

### EXAMPLE 5

Compounds Effects on ERK Substrate Phosphorylation

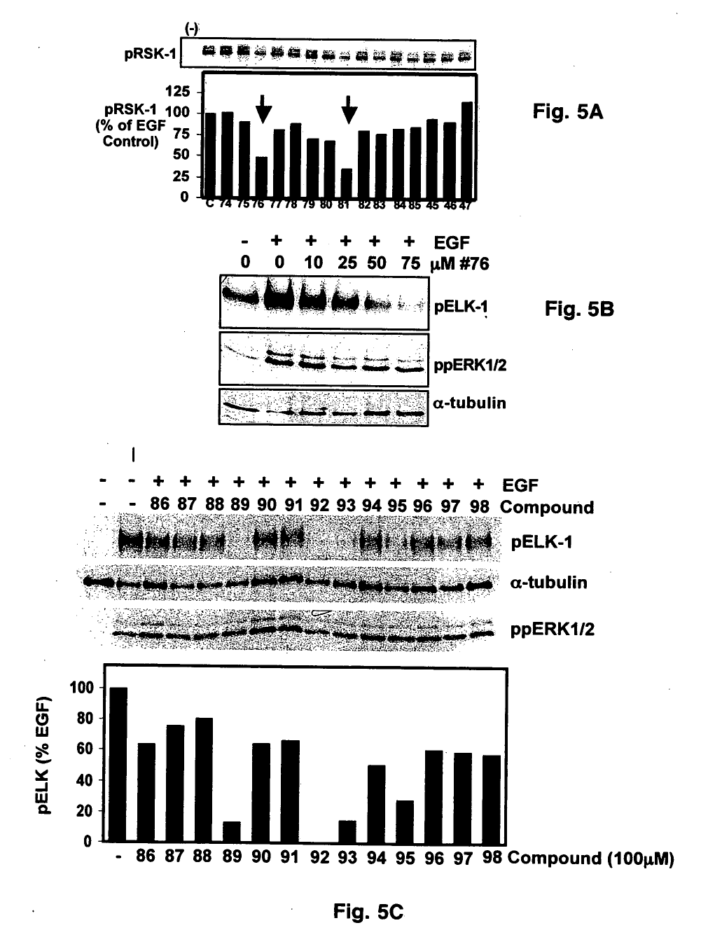

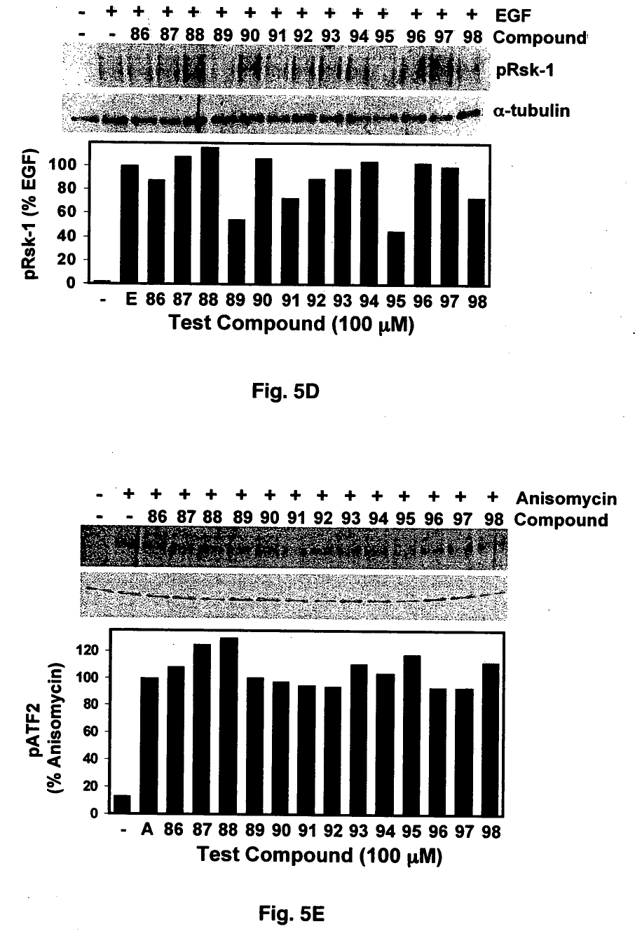

All compounds were subjected to assays of ERK specific phosphorylation of Rsk-1 and/or Elk-1 as examined by immunoblot analysis using phosphorylation specific antibodies. In FIG. 5A HeLa cells were cultured in 24 well plates and pretreated for 20-30 minutes with 0-100 mM of the selected test compounds. The cells were stimulated with epidermal growth factor (EGF, 50 ng/ml) for 5 minutes to activate the ERK pathway. Cell lysates were collected and immunoblotted for ERK-mediated phosphorylation of Rsk-1 on Thr573. As shown, EGF treatment alone caused a robust increase in Thr573 phosphorylation on Rsk-1 in the absence of test compounds. A typical immunoblot for Rsk-1 phosphorylation in the presence of 15 test compounds is shown in FIG. 5A. The presence of test compounds had inhibitory effects on ERK-mediated Rsk-1 phosphorylation. In these samples, densitometry quantification of the immunoblots showed that two compounds caused greater than 50% inhibition of Rsk-1 phosphorylation. Four additional compounds (17, 36, 79 and 80) inhibited ERK-mediated Rsk-1 phosphorylation by 20-25% out of the 80 compounds tested.

The ERK-specific phosphorylation of the transcription factor Elk-1 on Ser383 was also tested with the compounds that showed the highest inhibition of Rsk-1 phosphorylation in FIG. 5A, i.e. compound 76. As shown, increasing doses of compound 76 inhibited ERK-mediated Elk-i phosphorylation in response to EGF stimulation (FIG. 5B). As a protein loading control, the expression of α-tubulin was unchanged. Importantly, ERK1/2 phosphorylation on its activating sites was largely unaffected by the test compound. This finding support the specificity of this test compound for inhibiting ERK phosphorylation of downstream substrates, but has little effect on ERK protein phosphorylation by its upstream activator MEK1/2.

Compounds 86-98 were first tested for inhibition of ERK mediated phosphorylation of the transcription factor Elk-1, which is an important regulator of cell proliferation. Cells were treated with EGF to activate the ERK pathway and phosphorylation of Elk-1 on the ERK site at S383 was detected by immunoblot analysis (FIG. 5C). The test compounds inhibited Elk-1 phosphorylation by 20-100% compared to the EGF control (FIG. 5C). Compounds 89, 92, 93, and 95 were most effective at inhibiting Elk-1 phosphorylation at 100 EM. The effects of the test compounds were also tested with Rsk-1, another ERK substrate involved in regulating cell proliferation. Compounds 89, 91, and 95 were the most effective at inhibiting ERK-mediated Rsk-1 phosphorylation (FIG. 5D).

While the inhibition of Rsk-1 can be explained by the test compounds designed to bind to the CD region, which is required for Rsk-1 interactions with ERK2, it is not entirely apparent why the test compounds would interfere with Elk-1 interactions, which are thought to use different ERK residues for interactions (73). The test compounds may bind to other regions of ERK proteins or may have allosteric effects. Such differential inhibitory specificity of the inhibitors indicates that the identification substrate specific inhibitors of ERK are feasible.

The test compounds were also tested for selectivity for ERK versus the p38 MAP kinase. The phosphorylation of ATF2, a transcription factor substrate for the p38 MAP kinase, was not affected by any of the test compounds (FIG. 5E). In addition, the test compounds did not affect ERK1 and ERK2 phosphorylation indicating that they do not inhibit the upstream ERK activating proteins, MEK1 or MEK2 (FIG. 5C). Thus, the active compounds demonstrate selectivity towards ERK.

### EXAMPLE 6

Interaction of Active Compounds with ERK2

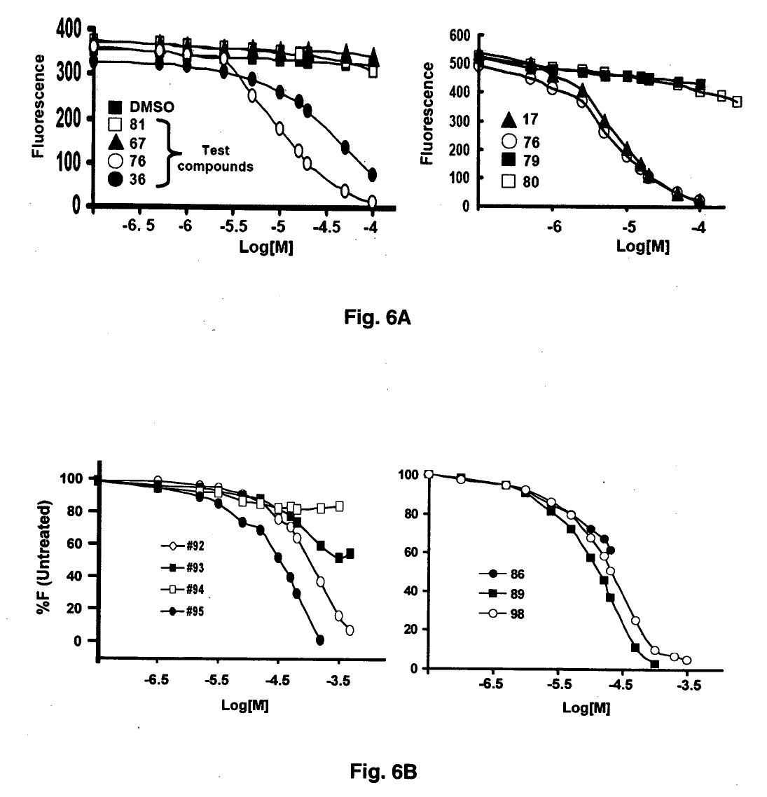

It was determined whether the active compounds directly interact with ERK2 using fluorescence spectroscopy. ERK2 contains three tryptophans, which have intrinsic fluorescence. Fluorescence quenching of ERK2 by the test compounds is indicative of binding interactions and potential protein conformational changes. Of the two compounds shown to be most active in all biological assays, 76 and 81, 76 shows strong quenching of fluorescence while quenching only occurs to a small extent at the higher concentrations with 81 (FIG. 6A).

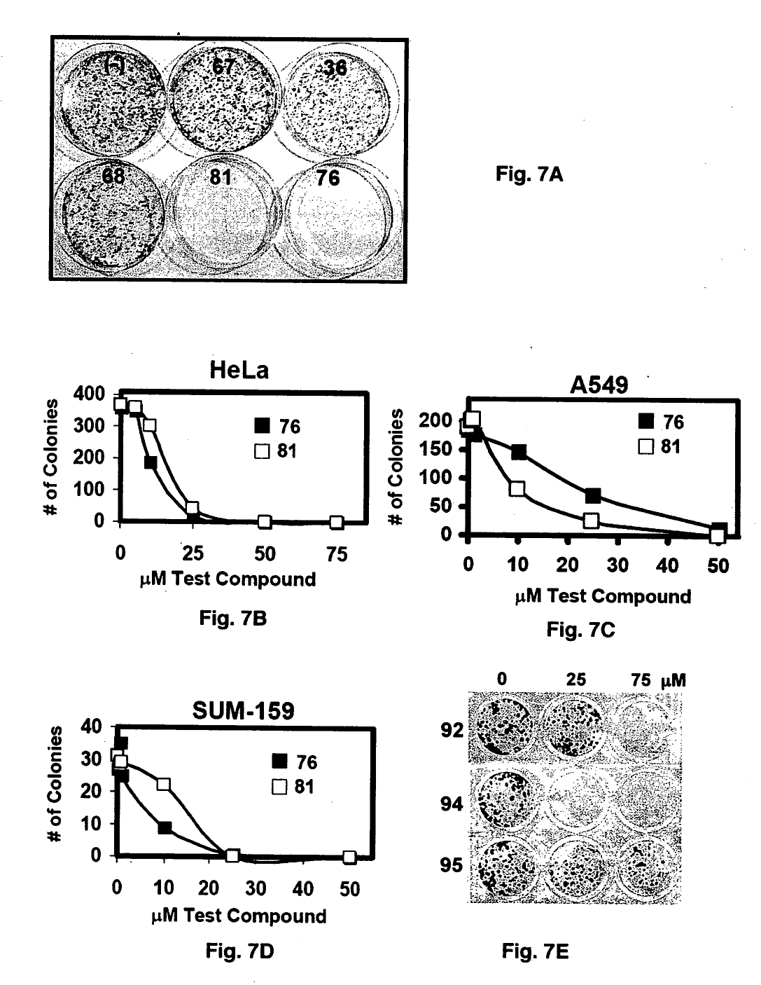

Compound 36 also showed significant quenching (FIG. 6A). Interestingly, compound 36, which also had little effect on ERK-mediated Rsk-1 phosphorylation but caused a subtle inhibition of colony formation (FIG. 7A below), showed significant binding with ERK2 (FIG. 6A). It is contemplated that compound 36 may be useful for future analysis of ERK function and substrate phosphorylation. In addition, compound 67, which significantly reduced RSK-1 phosphorylation also did not show quenching at the concentrations tested (FIG. 6A). Compound 68, which enhanced ERK-mediated RSK-1 phosphorylation showed strong auto-fluorescence in the absence of ERK2 protein. Thus, these assays could not determine whether compound 68 was interacting with ERK2. X-ray crystallography, as discussed in Example 1, may help determine the interactions between compounds that auto-fluoresce and ERK2.

From the fluorescence titrations, via reciprocal plots, K_{D} values of 5 and 16 mM were calculated for 76 and 36, respectively, with y-intercepts of 1.8 and 1.1, respectively, indicating a single binding site on the protein. Thus, the fluorescence quenching experiments indicate that 76 is binding directly to ERK2, thereby leading to its biological activity. Importantly, the K_{D} for compound 76, as determined from the fluorescence quenching, is similar to the approximate IC₅₀ determined based on colony formation (FIG. 7A below). Compound 17, which also inhibited colony formation, had a similar K_{D} as 76 (FIG. 6A). These findings suggest that any biological effects of 17, 36, and 76 are ERK-mediated while the effects of compounds 67, 79, 80, and 81 on ERK phosphorylation may not be via ERK-specific interactions.

The effects on ERK2 fluorescence were tested using the compounds that were identified to disrupt substrate interactions with the CD and ED domain using the phosphorylated ERK2 structure. Compounds 92 and 95 were effective in quenching ERK2 fluorescence with KD values of approximately 45 and 16 μM, respectively (FIG. 6B). Compound 93, which caused some quenching of ERK2 fluorescence, underwent auto-fluorescence at higher concentrations (FIG. 6B). The lack of fluorescence quenching by compound 94 suggested that this molecule did not bind ERK2 and that its effects on substrate phosphorylation and cell proliferation were potentially non-specific. Additional test compounds, including 89 and 98, also caused fluorescence quenching indicative of interactions with ERK2 with an approximate KD of 13 and 20 μM, respectively (FIG. 6B). Although compound 86 also caused some fluorescence quenching at concentrations of 20 μM or less, this compound became insoluble at concentrations of 50 μM or higher.

To determine whether the compounds bind specifically to the region identified by CADD, point mutations were generated in the CD or ED regions of ERK2 and tested to determine whether compound binding to ERK2 via fluorescence titrations is altered. Both point mutations, Thr157 to alanine (T157A) in the ED domain and Asp316 to asparagine (D316N) in the CD domain, tested with compound 76 showed an approximately 5 fold reduction in binding affinity based on fluorescence quenching as compared to wild type ERK2 (data not shown). These data indicate that changes in the binding pocket targeted by CADD disrupt compound binding providing evidence that the compounds are targeting the region of the ED and CD domain.

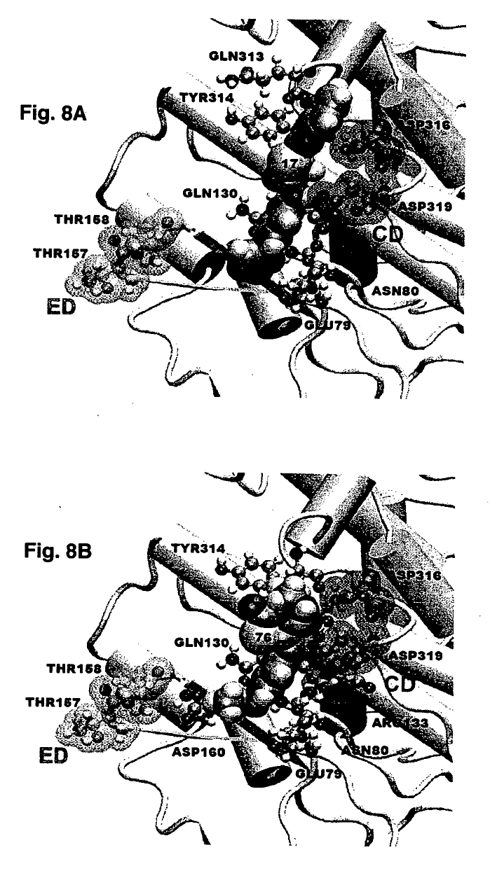

Alternatively, other amino acids depicted in FIGS. 8A-8B can be mutated using site directed mutagenesis (74) to characterize the CD and ED domain. Additional ERK2 mutants, containing a threonine to alanine mutation at residue 158, and an aspartate to alanine mutation at residue 319, may be generated. Moreover, ERK2 mutants at the other docking sites listed in Table 2 can be generated depending on the outcome of the CADD and substrate phosphorylation analysis. The fluorescence intensity is determined using ERK2 mutants incubated with the active compounds at the concentrations described above and compared with the fluorescence intensity determined using wild type ERK2. If an amino acid residue is important for the structure of a particular docking groove and binding of the test compounds, then fluorescence quenching is diminished in the ERK2 mutants as compared to ERK2 wild type. To control for the possibility that docking site mutations may cause structural changes that affect catalytic activity, mutant and wild type ERK2 enzymatic activity may be compared in cells and in vitro.

### EXAMPLE 7

Effects of Active Compounds on Cell Proliferation

The effects of the active compounds on cell proliferation and survival were tested using a colony formation assay. A screen of five test compounds showed that two compounds (76 and 81) completely inhibited cell proliferation, as evident by decreased number of cell colonies (FIG. 7A). Other compounds, including 36, 67, and 68, had little effect on colony formation (FIG. 7A).

Dose response assays demonstrated that compounds 76 and 81 similarly inhibited HeLa cell colony formation with an IC₅₀ of approximately 15-20 μM (FIG. 7B). In A549 lung carcinoma cells the IC₅₀ for compounds 76 and 81 was approximately 25 and 15 μM, respectively (FIG. 7C). Moreover, inhibition of cell proliferation following incubation with compounds 76 and 81 was observed in the SUM-159 estrogen-receptor negative breast cancer cell line (FIG. 8D) and HT1080 fibrosarcoma cells (data not shown). Compounds 17, 79, and 80 also inhibited HeLa, A549, HT1080, and MDA-MB-468 cell proliferation with IC₅₀ values similar to 76 and 81. Thus, it is contemplated that the compounds of the present invention exhibit inhibition of ERK substrates and of cancer cell proliferation, as demonstrated herein in vitro.

The IC50 concentration (micromolar) of compounds 86-98 was determined as shown in Table 3. HeLa cells were plated at low density and incubated with a single dose (0-100 μM) of the test compounds being tested. After 8-10 days, the resulting cell colonies were stained, photographed, and counted to estimate the IC50. The data is derived from at least 3 independent experiments. Test compounds 86 and 89 were the most effective inhibitors of cancer cell proliferation with IC50 values of 5 μM or less (Table 1). Test compounds 93, 94, and 95 were also effective inhibitors of cancer cell proliferation with IC50 values in the 5-10, 25-50, and 10-25 μM range, respectively. Although test compound 92 was a potent inhibitor of Elk-1 phosphorylation, the IC50 in the growth assay was approximately 75 μM). Test compound 89 was the most effective in inhibiting both Elk-1 and Rsk-1 phosphorylation and cancer cell proliferation.

TABLE 3

| Compound | IC₅₀(μM) |
|---|---|
| 86 | ˜5 |
| 87 | >100 |
| 88 | >100 |
| 89 | <2 |
| 90 | >100 |
| 91 | >100 |
| 92 | ˜75 |
| 93 | 5-10 |
| 94 | 25-50 |
| 95 | 10-25 |
| 96 | >100 |
| 97 | >100 |
| 98 | >100 |

Compounds 86-98 were tested in a colony formation assay. Colony formation inhibition is shown for compounds 92 and 94-95. All compounds showed some degree of colony formation inhibition (FIG. 7E), although compound 94 and 93 were the most effective inhibitors of colony formation, the effect may be non-specific as these compounds interactions with ERK2 were inconclusive as determined by fluorescence titrations (FIG. 6B). However, 92 and 95 inhibited in the 10-100 mM range, consistent with the binding in the fluorescence quenching experiment (FIG. 6B), indicating their function to be associated with direct binding to ERK. The differences in effects of active compounds on cell proliferation may be due to differences in how the active compounds affect ERK substrate phosphorylation. For example, active compounds that show stronger inhibition of cell proliferation may target a broader range of ERK substrates.

### EXAMPLE 8

Predicted Structures of Ligand-ERK2 Complexes

As the experimental fluorescence results confirm that compounds 17 and 76 bind to directly ERK2, it is of interest to understand the nature of the interactions between those compounds and ERK2. A detailed atomic picture of the predicted binding modes for these compounds identified from the screen using the unphosphorylated ERK2, as described in Example 4, is presented in FIGS. 8A-8B. Based on these predicted binding conformations, the compounds fit nicely into the groove that is located between the ED and CD sites. With both compounds, binding is predicted to occur adjacent to the CD site which places the compounds approximately 5-7 Å away from the threonine residues of the ED site, which forms a small protrusion on the protein surface.

The groove into which the compounds bind is polar containing several charged amino acids that are involved in multiple favorable interactions with the compounds. ERK2 residues with atoms within 3 Å of the compounds were Glu79, Asn80,Gln130, Arg133, Tyr314, Gln313, and the two aspartates from the CD site, Asp316 and Asp319. Several hydrogen bonds are observed between the aspartates and 17 and 76 (FIGS. 8A-8B). Arg133 is located above the aromatic rings in 76 and 17 potentially forming a cation-pi bond. Tyr314 makes a CH—O interaction through its backbone oxygen with 76. In addition, if the protein structure was allowed to relax around the bound compound, it is likely that more inhibitor-ERK2 interactions would be identified. Thus, based on the predicted binding interactions, one or more inhibitor-protein interactions can contribute to the binding affinity and/or the specificity for the ERK2 protein.

### EXAMPLE 9

Effects of Test Compounds on JNK and p38 MAP Kinase Substrates

The effects of test compounds on the JNK- or p38-specific substrates are tested. Table 1 above includes some of the available phospho-specific antibodies against JNK and p38 substrates. Since the docking domains that are targeted in ERK2 may share features with the p38 MAP kinases (34), it is determined whether the biologically active compounds target substrates that can be phosphorylated by both kinases. As one example, ERK and p38 dually phosphorylate the MAP kinase integrating kinase-1 (MNK-1) on the same threonine sites at positions 197 and 202 (75). Similarly, JNK and p38 may also target S383 on ELK-1. Compounds are tested for specificity to ERK, JNK or p38 substrate phosphorylation by treating cells with factors known to specifically activate each pathway. Cells are treated with epidermal growth factor (EGF) or anisomycin to activate ERK or p38, respectively. JNK activity can be specifically activated by over-expression of MLK3 (45). This determines whether the active compounds can selectively discriminate between the various MAP kinase substrates.

ERK or p38 activity in the context of treatment with candidate compounds is examined. HeLa cells are transfected with constitutively active mutants of MEK1, which only activates ERK proteins (76) or MEK3, which primarily activates p38 but not ERK (77). Transfected cells are incubated in the absence or presence of biologically active compounds and ERK2 or p38 substrate phosphorylation is determined by immunoblotting. It is contemplated that biologically active compounds that target p38 may have additional utility for the development of new molecules aimed at treating inflammatory diseases (78).

### EXAMPLE 10

In vitro Experiments with Active ERK Incubated with Specific Substrates

In vitro kinase assays are done using purified active ERK2 (commercially available or generated as described in Example 1) incubated with specific substrates, ELK-1 and c-Myc (generated by expression vectors, for example) or a non-specific myelin basic protein (MBP) peptide in the presence of 0, 1, 5, 10, 20, 30, 40, 50, and 75 mM of the test compounds showing biological activity. The MBP peptide, which does not require the CD or ED domain in order to be phosphorylated by ERK, is used as a control for measuring the effects of the test compounds on ERK2 catalytic activity. ELK-1 or c-Myc substrate phosphorylation is measured by phosphorimager analysis following gel electrophoresis and expressed as a ratio of the MBP phosphorylation under each test drug concentration.

Although data suggest that ERK activity is not affected by the test compounds, cell based experiments are performed to confirm these observations. HeLa cells are stimulated with EGF in the presence or absence of test compounds (0, 25, 50 or 100 mM) and ERK2 is immunoprecipitated for kinase assays done in the presence of radiolabeled ATP and the non-specific substrate MBP. MBP phosphorylation is measured by scintillation counting. It is contemplated that immunoprecipitated EGF-stimulated ERK2 phosphorylation of MBP is not affected in the presence of the test compounds. As a control for the cell based and in vitro experiments, the general kinase inhibitor, staurosporine, is used to inhibit ERK2 activity.

Alternatively, the efficacy of the test compounds, identified using the ERK2 structure, for binding to ERK1 is determined using fluorescence titration assays. Whereas, the corresponding residues surrounding the ED domain are identical in ERK1 and ERK2, the CD domain is different as shown in the sequences below of the amino acids surrounding the CD domain region of ERK2 and the corresponding region in ERK1. The underlined amino acids are different between ERK1 and ERK2 in the CD domain.

| | | |
|---|---|---|
| ERK2: | PYLEQYYD₃₁₆PSD₃₁₉EPIAEA | (SEQ ID NO: 3) |
| ERK1: | PYLEQYYD₃₃₆PTD₃₃₉EPVAEE | (SEQ ID NO: 4) |

It is recognized that the test compounds may have effects on other MAP kinases, which are less well characterized. For example, chemical inhibitors of MEK1/2 may also inhibit the activity of the MEK5/ERK5 signaling pathway (79). In addition, consideration must be given to other kinases that are not related to MAP kinases but also play a role in the survival of cancer cells. For example, the serine/threonine kinase Akt has been implicated in promoting cancer cell survival (80). Once a candidate compound is identified, a comprehensive examination of its effects on multiple families of kinases are conducted using the antibody microarray in Example 1.

### EXAMPLE 11

Pharmacokinetics of Test Compounds

The cellular metabolism of the test drugs is assessed in the HeLa, MDA-MB-468, SUM159, HCT116, and SK-Mel-28 cell lines. The cellular metabolic profile and kinetics of test compound is be determined using the cells in vitro. To determine if changes in intracellular test compound and metabolite concentrations are relevant, the intratumoral pharmacokinetics of the test compound and its metabolites are assessed in tumor bearing mice as in Example 1.

The test compounds and metabolites are quantified using a high performance liquid chromatography (HPLC) assay with ultraviolet (UV), fluorescence detection, or tandem mass spectrometry detection (LC/MS/MS) (81-83). Metabolite identification is verified using a modified liquid chromatography with triple quadrupole mass spectrometric detection (LC/MS/MS). After trypsinization, cells are plated onto 6 cm plates at a seeding density of a million cells per plate. After 24 hours, the cells are incubated with the test compounds at a concentration of 10-100 mM for 0, 5, 10, 30, or 60 minutes. The cells are harvested using 1N KOH and analyzed. Cellular uptake and kinetics of test compounds and major metabolites are measured in the cell lines using HPLC methods.

### EXAMPLE 12

In vivo Tumor Model

Human MDA-MB-468 breast cancer cells are initially used as a xenograft model in athymic nude mice (nu/nu, 5-6 week old: Harlan Sprague Dawley, Inc.). These cells are well established for developing tumors in this model. However, HCT116 and SK-Mel-28 have also been shown to cause tumors in nude mice and may be used.

The hind leg is an established model for establishing a xenograft whose growth parameters may be measured and modeled to existing data. The nude mice are implanted subcutaneously with 10⁶ MDA-MB-468 cells in 0.5 ml sterile saline as described (18) in the presence or absence of test compounds. Tumor growth is monitored daily by calipers. Alternatively, after tumors reach a mean diameter of 4-5 mm² in size, the animals are left untreated or treated with the test compounds prepared in a vehicle of 10% DMSO/saline as below. Statistical comparisons are made using a two sample student's T test to assess the mean difference in tumor size between the control and treated groups. Significance is defined as difference in tumor size resulting in a p value<0.05.

At the beginning of cancer cell injection or after the tumors reach a mean diameter of 4-5 mm² in size, the animals are intravenously injected via the lateral tail vein with the test compounds (50 mg/kg) or vehicle control once daily for two days. Three animals per time-point are sacrificed at each of the following timepoints: 0.25, 1, 4, and 8 hours after test compound administration. Control blood and tissue for all study groups is collected at approximately 0.25 hours after administration of vehicle alone.

Blood is collected via cardiac puncture from three animals at each timepoint. The blood is centrifuged at 4° C. at 1250×g for 10 minutes. Plasma is separated into cryotubes and stored at −80° C. Organs (liver, heart, brain, kidney, and tumor tissue) are removed and placed on dry ice, weighed and snap frozen in liquid nitrogen. The total number of animals for this study is 120. An additional 12 animals are used as untreated controls. The concentration of each test compound and its metabolites are measured via HPLC in mouse plasma, liver, heart, brain, kidney, and tumor tissue. Pharmacokinetic analysis is performed using a model independent approach as described in Example 1.

The following references are cited herein.
1. Pearson et al., 2001, Endocr Rev, 22:153-183.
2. Kyriakis, J. M. and Avruch, J., 1996, Bioessays, 18:567-577.
3. Reuter et al. 2000, Blood, 96:1655-1669.
4. Duesbery et al., 1999, Nat Med 1999, 5:736-737.
5. Shapiro, P. 2002, Crit Rev Clin Lab Sci 2002, 39:285-330.
6. Cohen, P., 1999, Curr Opin Chem Biol 1999, 3:459-465.
7. Lewis et al., 1998, Advances in Cancer Research 1998, 74:49-139.
8. Zhang et al., 1995, Structure 1995, 3:299-307.
9. Bos J. L., 1989, Cancer Res 1989, 49:4682-4689.
10. Brose et al., 2002, Cancer Res 2002, 62:6997-7000.
11. Songyang et al., 1996, Mol Cell Biol, 16:6486-6493.
12. Zuniga et al., 1999, J Biol Chem, 274:21900-21907.
13. Fantz et al., 2001, J Biol Chem, 276:27256-27265.
14. Zhang et al., 2003, J Biol Chem, 278:29901-29912.
15. Nichols et al., 2000, J Biol Chem, 275:24613-24621.
16. Tanoue et al., 2000, Nat Cell Biol, 2:110-116.
17. Bollag et al., 2003, Curr Opin Investig Drugs, 4:1436-1441.
18. Boldt et al., 2004, Curr Pharm Des, 10:1885-1905.
19. Sebolt-Leopold, J. S., 2004, Curr Pharm Des, 10:1907-1914.
20. Allen et al., 2003, Semin Oncol, 30:105-116.
21. Bain etal., 2003, Biochem J, 371:199-204.
22. Gohlke et al., 2002, Angew Chem Int Ed Engl, 41:2644-2676.
23. Markowitz et al., 2004, J Med Chem, 47:5085-5093.
24. Loughney et al., 1999, Med. Chem. Res., 9:579-591.
25. Zeng et al., 2001, Protein Eng, 14:39-45.
26. Huang et al., 2004, J Med Chem, 47:3502-3511.
27. Zhang et al., 1994, Nature, 367:704-711.
28. Canagarajah et al., 1997, Cell, 90:859-869.
29. Bernstein et al., 1977, Eur J Biochem, 80:319-324.
30. Kuntz et al., 1982, J Mol Biol, 161:269-288.
31. Kuntz, I. D., 1992, Science, 257:1078-1082.
32. Connolly, M. L., 1983, Science, 221:709-713.
33. Ferrin etal., 1988, J. Mol. Graphics, 6:13-27.
34. Tanoue et al., 2001, Embo J, 20:466-479.
35. Goodford et al., 1984, J. Med. Chem., 28:849-857.
36. Pan et al., 2003, J Chem Inf Comput Sci, 43:267-272.
37. Leach et al., 1992, J. Comput. Chem., 13:730-748.
38. Ewing et al., 1997, J. Comput. Chem., 18:1175-1189.
39. Butina et al., 1999, J. Chem. Inf. Comp. Sci., 39:747-750.
40. Lipinski et al., 2001, Adv Drug Deliv Rev, 46:3-26.
41. Cha et al., 2001, J Biol Chem, 276:48494-48501.
42. Dangi et al., 2003, Cell Signal, 15:667-675.
43. Cha et al., 2004, Biochem J, 378:857-865.
44. Dangi, S., and Shapiro, P., 2005, J Biol Chem, 280:24524-24531
45. Chan et al., 2004, Nat Med, 10:1390-1396
46. Shapiro et al., 1999 Mol Cell Biol, 19:3551-3560.
47. Lakowicz, J. R., Principles of Fluorescence Spectroscopy. Kluwer Academic/Plenum Publishers Second Edition 1999.
48. Marshall, G., Biophysical Chemistry: Principles, Techniques, and Applications. John Wiley & Sons: New York 1978.
49. MacKerell et al., 1991, Biochim Biophys Acta, 1073:357-365.
50. Chang et al., 2002, Mol Cell, 9:1241-1249.
51. Zhang et al., 1993, Journal of Molecular Biology 233:550-552.
52. Bailer, A. J., 1988, J Pharmacokinet Biopharm, 16:303-309.
53. Robinson et al., 2002, J Biol Chem, 277:5094-5100.
54. Hoofnagle et al., 2001, Proc Natl Acad Sci U S A. 98(3):956.
55. Meng et al., 1994, J Comput Aided Mol Des 8:299-306.
56. Milne et al., 1994, J Chem Inf Comput Sci,34:1219-1224.
57. Hayashi and MacKerell, Jr., 2001, Immunomodulatory Compounds, USA.
58. Teague et al., 1999, Angew Chem Int Ed Engl, 38:3743-3748.
59. Nicklaus et al., 1997, J Med Chem, 40:920-929.
60. MacKerel, et al., 1998, Encyclopedia of Computational Chem. 1:271-277.
61. Brooks et al., 1983, J. Comput. Chem. 4:187-217.
62. Bissantz et al., 2000, J Med Chem, 43:4759-4767.
63. Charifson et al., 1999, J Med Chem, 42:5100-5109.
64. Gohlke, H., and Klebe, G., 2001, Curr Opin Struct Biol, 11:231-235.
65. Muegge, I., and Martin, Y. C., 1999, J Med Chem, 42:791-804.
66. Chen, X., and Tropsha, A., 1999, J. Comput. Chem., 20:749-759.
67. Aqvist et al., 1994, Protein Eng, 7:385-391.
68. Qui et al., 1997, J. Phys. Chem., 101:3005-3014.
69. Lee et al., 2003, J Comput Chem, 24:1348-1356.
70. Feig et al., 2004, J Comput Chem, 25:265-284.
71. Oprea, et al., 2001, J Chem Inf Comput Sci, 41:1308-1315.
72. Hancock et al., 2005., J Med Chem. 48(14),:4586.
73. Dimitri et al., 2005, Curr Biol. 15(14):1319.
74. Cha, H., and Shapiro, P., 2001, J Cell Biol, 153:1355-1368.
75. Waskiewicz et al., 1997, Embo J, 16:1909-1920.
76. Mansour et al., 1996, Cell Growth Differ, 7:243-250.
77. Raingeaud et al., 1996, Mol Cell Biol, 16:1247-1255.
78. Herlaar, E., and Brown, Z., 1999, Mol Med Today, 5:439-447.
79. Mody et al., 2001, FEBS Lett, 502:21-24.
80. Datta et al., 1999, Genes Dev, 13:2905-2927.
81. Simmons et al., 1997, Analytical Letters, 30:2009-2021.
82. Rudek et al., 2000, J Pharm Biomed Anal, 22:1003-1014.
83. Bauer et al., 2000, Biomed Chromatogr, 14:338-343.

Any patents or publications mentioned in this specification are indicative of the levels of those skilled in the art to which the invention pertains. Further, these patents and publications are incorporated by reference herein to the same extent as if each individual publication was specifically and individually incorporated by reference. One skilled in the art will appreciate that the present invention is well adapted to carry out the objects and obtain the ends and advantages mentioned, as well as those objects, ends and advantages inherent herein. Changes therein and other uses which are encompassed within the spirit of the invention as defined by the scope of the claims will occur to those skilled in the art.

What is claimed is:

1. A method of inhibiting an activity of an extracellular signal-regulated kinase (ERK) in a cell, comprising:
contacting the cell with an inhibitory compound that selectively binds to one or more docking domain regions of said extracellular signal-regulated kinase thereby inhibiting an extracellular signal-regulated kinase activity associated with an extracellular signal-regulated kinase substrate binding thereto.

2. The method of claim 1, wherein said extracellular signal-regulated kinase is ERK1 or ERK2.

3. The method of claim 1, wherein said docking domain region(s) comprises one or more of a CD domain, an ED domains, a SB domain, or a MS domain.

4. The method of claim 1, wherein said inhibitory compound is compound 17, compound 36, compound 76, compound 79, compound 80, compound 81, or one of compounds 86-98.

5. The method of claim 1, wherein said cell is a neoplastic cell.

6. The method of claim 5, wherein said neoplastic cell comprises a breast cancer, a lung cancer, a cervical cancer, a pancreatic cancer, a bladder cancer, a colon cancer, or a cancer having a Ras mutation.

7. A method of inhibiting proliferation of a neoplastic cell, comprising:
contacting the neoplastic cell with an inhibitory compound that selectively inhibits binding of a substrate of an extracellular signal-regulated kinase to one or more docking domain regions thereof whereby proliferation of the neoplastic cell is inhibited; wherein said inhibitory compound is compound 17, compound 76, compound 86, compound 89, compound 92, compound 93, compound 94, or compound 95.

8. The method of claim 7, wherein said extracellular signal-regulated kinase is ERK2.

9. The method of claim 7, wherein said docking domain region(s) comprises one or more of a CD domain, an ED domains, a SB domain, or a MS domain.

10. The method of claim 7, wherein said neoplastic cell comprises a cancer selected from the group consisting of a breast cancer, a lung cancer, a cervical cancer, a pancreatic cancer, a bladder cancer, a colon cancer, or a cancer having a Ras mutation.

11. A method of treating a cancer in a subject, comprising:
administering an inhibitory compound that selectively binds to one or more docking domain regions of an extracellular signal-recognition kinase to reduce proliferation of cells comprising the cancer upon binding said inhibitory compound thereto, thereby treating the cancer in the subject.

12. The method of claim 11, further comprising:
administering an anticancer drug to the subject.

13. The method of claim 12, wherein said anticancer drug is administered concurrently or sequentially with the inhibitory compound.

14. The method of claim 11, wherein said extracellular signal-regulated kinase is ERK1 or ERK2.

15. The method of claim 11, wherein said docking domain region(s) comprises one or more of a CD domain, an ED domains, a SB domain, or a MS domain.

16. The method of claim 11, wherein said inhibitory compound is compound 17, compound 36, compound 76, compound 79, compound 80, compound 81, or one of compounds 86-98.

17. The method of claim 16, wherein said inhibitory compound is compound 17, compound 76, compound 86, compound 89, compound 92, compound 93, compound 94, or compound 95.

18. The method of claim 11, wherein said anticancer drug is cisplatin, oxaliplatin, carboplatin, doxorubicin, a camptothecin, paclitaxel, methotrexate, vinblastine, etoposide, docetaxel hydroxyurea, celecoxib, fluorouracil, busulfan, imatinib is mesylate, alembuzumab, aldesleukin, or cyclophosphamide.

19. The method of claim 11, wherein a dosage of said anticancer drug is lower than a dosage required when said anticancer drug is administered singly, thereby reducing toxicity of the anticancer drug to the individual.

20. The method of claim 11, wherein said cancer is a breast cancer, a lung cancer, a cervical cancer, a pancreatic cancer, a bladder cancer, a colon cancer, or a cancer having a Ras mutation.

21. A method of reducing toxicity of a cancer therapy in an individual in need thereof, comprising:
administering to the individual an inhibitory compound that selectively binds to one or more docking domain regions of an extracellular signal-recognition kinase and an anticancer drug, wherein a dosage of the anticancer drug administered with the inhibitory compound is lower than a dosage required when said anticancer drug is administered singly, thereby reducing toxicity of the cancer therapy to the individual.

22. The method of claim 21, wherein said anticancer drug is administered concurrently or sequentially with the inhibitory compound.

23. The method of claim 21, wherein said extracellular signal-regulated kinase is ERK2.

24. The method of claim 21, wherein said docking domain region(s) comprises one or more of a CD domain, an ED domains, a SB domain, or a MS domain.

25. The method of claim 21, wherein the inhibitory compound is compound 17, compound 36, compound 76, compound 79, compound 80, compound 81, or one of compounds 86-98.

26. The method of claim 25, wherein said inhibitory compound is compound 17, compound 76, compound 86, compound 89, compound 92, compound 93, compound 94, or compound 95.

27. The method of claim 21, wherein said anticancer compound is cisplatin, oxaliplatin, carboplatin, doxorubicin, a camptothecin, paclitaxel, methotrexate, vinblastine, etoposide, docetaxel hydroxyurea, celecoxib, fluorouracil, busulfan, imatinib mesylate, alembuzumab, aldesleukin, and cyclophosphamide.

28. The method of claim 21, wherein said cancer is a breast cancer, a lung cancer, a cervical cancer, a pancreatic cancer, a bladder cancer, a colon cancer, or a cancer having a Ras mutation.

29. A method of identifying an inhibitor of substrate binding to a docking domain region of an extracellular signal-reduction kinase, comprising:
designing a test compound that binds to one or more docking domain regions in extracellular signal-regulated kinase, but does not interfere with the ATP binding domain, wherein said design is based at least in part on computer-aided drug design modeling;
measuring the level of phosphorylation of a extracellular signal-regulated kinase substrate protein in the presence or absence of the test compound; and
comparing the level of protein phosphorylation in the presence of the test compound with the level of protein phosphorylation in the absence of the test compound, wherein a decrease in protein phosphorylation in the presence of the test compound is indicative that the test compound is an inhibitor of binding to one or more docking domain regions in the extracellular signal-regulated kinase.

30. The method of claim 29, wherein said extracellular signal-regulated kinase is ERK2.

31. The method of claim 29, wherein said docking domain region(s) comprises one or more of a CD domain, an ED domains, a SB domain, or a MS domain.

32. The method of claim 31, wherein said inhibitor binds with residues Asp316, Asp319 or a combination thereof comprising the CD domain and with at least one of residues Glu79, Asn80,Gln230, Arg133, Tyr314, Gln313 comprising the ED domain.

33. The method of claim 29, further comprising:
screening said inhibitor for anti-cell proliferative activity directed against neoplastic cells.

34. The method of claim 33, wherein said screening step comprises:
contacting a culture of the neoplastic cells having an activated extracellular signal-regulated kinase activity with the inhibitor; and
comparing the amount of cell proliferation of the neoplastic cells in the presence of the inhibitor with the amount of cell proliferation of the neoplastic cells in the absence of the inhibitor, wherein a decrease in cell proliferation in the presence of the inhibitor compared to cell proliferation in the absence of the inhibitor is indicative that the inhibitor has the ability to prevent cell proliferation in the neoplastic cells.

35. The method of claim 34, wherein said neoplastic cell comprises a cancer.

36. The method of claim 35, wherein said cancer is a breast cancer, a lung cancer, a cervical cancer, a pancreatic cancer, a bladder cancer, a colon cancer, or a cancer having a Ras mutation.


SEQUENCE LISTING (4 sequences)

SEQ ID NO: 1 - protein, 346 aa, artificial sequence
  29..374: amino acids 29 to 374 of extracellular signal- related kinase 1 (ERK1)
EMVKGQPFDVGPRYTQLQYIGEGAYGMVSSAYDHVRKTRVAIKKISPFEHQTYCQRTLREIQILLRFRHENVIGIRDILRASTLEAMRDVYIVQDLMETDLYKLLKSQQLSNDHICYFLYQILRGLKYIHSANVLHRDLKPSNLLINTTCDLKICDFGLARIADPEHDHTGFLTEYVATRWYRAPEIMLNSKGYTKSIDIWSVGCILAEMLSNRPIFPGKHYLDQLNHILGILGSPSQEDLNCIINMKARNYLQSLPSKTKVAWAKLFPKSDSKALDLLDRMLTFNPNKRITVEEALAHPYLEQYYDPTDEPVAEEPFTFAMELDDLPKERLKELIFQETARFQPG

SEQ ID NO: 2 - protein, 346 aa, artificial sequence
  11..357: amino acids 11 to 357 of extracellular signal- related kinase 2 (ERK2)
EMVRGQVFDVGPRYTNLSYIGEGAYGMVCSAYDNVNKVRVAIKKISPFEHQTYCQRTLREIKILLRFRHENIIGINDIIRAPTIEQMKDVYIVQDLMETDLYKLLKTQHLSNDHICYFLYQILRGLKYIHSANVLHRDLKPSNLLLNTTCDLKICDFGLARVADPDHDHTGFLTEYVATRWYRAPEIMLNSKGYTKSIDIWSVGCILAEMLSNRPIFPGKHYLDQLNHILGILGSPSQEDLNCIINLKARNYLLSLPHKNKVPWNRLFPNADSKALDLLDKMLTFNPHKRIEVEQALAHPYLEQYYDPSDEPIAEAPFKFDMELDDLPKEKLKELIFEETARFQPG

SEQ ID NO: 3 - protein, 17 aa, artificial sequence
  309..325: CD domain of ERK1
PYLEQYYDPSDEPIAEA

SEQ ID NO: 4 - protein, 17 aa, artificial sequence
  329..345: CD domain of ERK2
PYLEQYYDPSDEPIAEA